(19) United States
(12) Patent Application Publication
(10) Pub. No.: US 2004/0126792 A1
(43) Pub. Date: Jul. 1, 2004

(54) Synthesis and applications of trinucleotide pCpCpA-3'-NH-aminoacyl derivatives

(76) Inventor: Biliang Zhang, Shrewsbury, MA (US)

Correspondence Address: FISH & RICHARDSON PC, 225 FRANKLIN ST, BOSTON, MA, 02110, US

(21) Appl. No.: 10/666,628

(22) Filed: Sep. 17, 2003

Related U.S. Application Data
(60) Provisional application No. 60/411,649, filed on Sep. 18, 2002.

(51) Int. Cl. C12Q 1/68; C07H 21/04

(52) U.S. Cl. 435/6; 536/24.3

(57) ABSTRACT

The present invention relates to the synthesis of novel compounds for the inhibition of peptide synthesis in the ribosome. It also relates to improved methods for directly monitoring peptide bond formation in the ribosome. These compounds can be used for high throughput screening of proposed therapeutically active drug species (e.g., antibiotics) targeted to ribosomes.

### CROSS-REFERENCE TO RELATED APPLICATIONS

This application claims priority to U.S. Provisional Application No. 60/411,649 filed on Sep. 18, 2002, the contents of which is incorporated herein by reference in its entirety.

### TECHNICAL FIELD

The present invention relates to the synthesis of novel compounds for the inhibition of peptide synthesis in the ribosome. It also relates to improved methods for directly monitoring peptide bond formation in the ribosome. These compounds can be used for high throughput screening of proposed therapeutically active drug species (e.g., antibiotics) targeted to ribosomes.

### BACKGROUND

Ribosomes are cytoplasmic organelles that serve as the catalyst of protein synthesis within all forms of life. The ribosome, a two subunit macromolecular complex, comprised in bacteria of three large RNA's (ribosomal RNA's or rRNA's) and more than 50 proteins, is recognized as the catalyst and framework of translation. Ribosomes translate messenger RNA (mRNA) into a polypeptide chain having a specific sequence. Peptidyl transferase (PT) is involved in the process by which peptide bonds are formed.

It has been known for over forty-five years that the peptidyl transferase activity responsible for the peptide bond formation that occurs during mRNA-directed protein synthesis is intrinsic to the large ribosomal subunit, and it has been understood for even longer that the ribosome contains proteins as well as RNA. In bacteria, for example, the large ribosomal subunit contains approximately thirty-five different proteins and two RNA's. By 1980, the list of components that might be part of the ribosome's peptidyl transferase center had been reduced to about a half dozen proteins and 23S rRNA. Following the discovery of catalytic RNA's, the hypothesis that 23S rRNA might be its sole constituent began to gain favor. In 1984, Noller and colleagues published affinity-labeling results that showed that U2619 and U2620 are adjacent to the CCA-end of the P-site bound transfer RNA (tRNA). These nucleotides are part of a highly conserved internal loop in the center of domain V of 23S rRNA. The hypothesis that this loop is intimately involved in peptidyl transferase activity is supported by the observation that mutations in the loop render cells resistant to many inhibitors of peptidyl transferase, and evidence implicating it in this activity has continued to mount. Recently, X-ray crystal structures of ribosomes illustrated that peptide bond formation (peptidyl transfer) is catalyzed by the 23S rRNA, although its mechanism is still unclear. (Nissen et al., Science, 289:905-930 (2000)). Nissen has shown that nucleotide bases, rather than the phosphodiester backbone, are the components of the ribosome closest to the site of peptide bond formation. Also, it has been postulated that the active-site region for peptidyl transferase activity is a particular adenosine, A2451, conserved in thousands of sequenced rRNA's, that plays a crucial role as a general acid-base catalyst. Muth, et al., Science, 289:947-950 (2000).

The sequence CCA, which is located at the 3′-terminal sequence of all tRNA's, binds to the large ribosomal subunit by itself, consistent with biochemical data showing that the interactions between tRNA's and the large subunit largely depend on their CCA sequences.

The 2′(3′)-O-aminoacyl-pCpCpA derivatives are the universally conserved terminal sequences of aminoacyl-tRNA and potential substrates for ribosomal peptidyl transferase. Evidence from crosslinking and chemical footprinting experiments has suggested specific tRNA:rRNA interactions, in which the universally conserved CCA of the 3′-end of tRNA and its attached aminoacyl moiety are involved in the interactions between tRNA and 23S rRNA. In most fragment reactions, CpCpA-fMet has been used as a peptidyl donor while puromycin (puromycin is an aa-tRNA analog that interacts with the ribosomal A-site, and the phosphoramide group of the Yarus compound mimics the tetrahedral carbon intermediate; this analog, CcdA-phosphate-puromycin (CcdA-p-Puro) binds tightly to the ribosome, and inhibits its peptidyl transferase activity) has been used as a peptidyl acceptor, as disclosed in U.S. Pat. No. 5,962,244, the disclosure of which is herein incorporated specifically by reference.

Despite decades of intensive research, the catalytic mechanism of protein synthesis in the ribosome is still largely unknown. This is not for lack of interest; indeed, investigators have expended much effort in studying this mechanism. However, investigations have been hampered by technical problems associated with the complexity of the ribosome and its substrates. Most assays of rRNA function involve in vitro reconstitution of 50S subunits and the peptidyl transferase (PT) activity is measured by either “fragment reaction” or “poly (Phe) synthesis” (Fahnestock et al., Methods Enzymol., 30:554-562 (1974). According to these prior art methods, reaction products of peptidyl transferase activity are analyzed by high-voltage paper electrophoresis, or by quantitative estimation of radioactivity in the ethyl acetate-extracted fraction by scintillation counting. The major drawback of these assays is that they do not directly observe peptide bond formation. Moreover, they have the additional limitations of having low sensitivity and requiring the addition of up to 30% alcohol.

Thus, there exists a need for assays of peptidyl transferase activity that overcome the deficiencies in the prior art, namely the low sensitivity and the requirement for addition of alcohol, among others. An improved system would exhibit higher sensitivity and would enable the direct monitoring of the progress of these reactions.

Moreover, ribonucleoprotein complexes represent inviting targets for pharmacological intervention. First, there are differences in protein synthesis mechanisms between pathogenic organisms and humans that allow for greater selectivity in the inhibition of PT activity by antibacterial species. Secondly, their structures contain well-defined binding sites comprised of both RNA and proteins. The bacterial ribosome has been known as the receptor for antibiotics blocking protein synthesis since the discovery of streptomycin in the 1940s, but new antibacterials are urgently needed to overcome the problem of drug resistance that severely limits the effectiveness of the current arsenal of antibiotics.

A key step in characterizing the peptidyl transferase reaction was achieved by development of the so-called fragment reaction, wherein the P-site aminoacyl-tRNA is replaced by a small-molecule derivative (e.g., 5′-CAACCA-formyl methionine) and the A-site aminoacyl-tRNA is replaced by puromycin (which mimics the 3′ terminus of an aminoacyl-tRNA). During the course of the reaction, the amino group of puromycin forms a peptide bond with fMet to yield an fMet-puromycin product. Characterization of this simple reaction, which isolates this phase of the overall translation elongation cycle from other steps (e.g., binding of aminoacyl-tRNA's, release of free tRNA's, mRNA binding and translocation), allowed for the delineation of the protein and RNA components of the ribosome as well as cofactors required specifically for peptide bond formation during elongation. Further studies established the authenticity of the fragment reaction as a valid model of normal peptide bond formation reactions in vivo by demonstrating inhibition of this reaction by antibiotics that have been determined to act at this level in whole cells.

A large number of antibacterial agents, including many in current clinical use, inhibit protein synthesis in bacteria by interfering with essential functions of the ribosome. When ribosomal function is perturbed, protein synthesis may cease entirely or, alternatively, it may be sufficiently slowed so as to stop normal cell growth and metabolism. Differences between the prokaryotic 70S ribosomes (composed of 50S and 30S subunits) and the eukaryotic 80S ribosome (composed of 60S and 40S subunits) underlie the basis for the selective toxicity of many antimicrobial agents of this class. However, a limited subset of this class of antimicrobial agents exhibits some cross-reactivity with the 70S ribosomes of eukaryotic mitochondria. This cross-reactivity probably accounts for the host cell's cytotoxicity effects observed with some agents and has limited their use as clinical antimicrobial agents. Other agents (e.g., tetracycline), which affect the function of eukaryotic 80S ribosomes in vitro, are still used clinically to treat bacterial infections as the concentrations employed during antimicrobial therapy are not sufficient to elicit host cell toxicity side-effects.

Moreover, protein biosynthesis inhibitors can be divided into a number of different classes based on differences in their mechanisms of action. The aminoglycoside agents (e.g., streptomycin) bind irreversibly to the 30S subunit of the ribosome, thereby slowing protein synthesis and causing mis-translation (i.e., mis-reading) of the mRNA. The resulting errors in the fidelity of protein synthesis are bacteriocidal, and the selective toxicity of this family of agents is increased by the fact that bacteria actively transport them into the cell. The tetracycline family of agents (e.g., doxycycline) also binds to the 30S ribosome subunit, but does so reversibly. Such agents are bacteriostatic and act by interfering with the elongation phase of protein synthesis by inhibiting transfer of the amino acid moieties of the aminoacyl-tRNA substrates into the growing polypeptide chain. However, inhibition mediated by the tetracyclines is readily reversible, with protein synthesis resuming once intracellular levels of the agents decline. Chloramphenicol and the macrolide family of agents (e.g., erythromycin), in contrast, act on the finction/activity of the 50S subunit of the ribosome. These agents are bacteriostatic in nature, and their effects are reversible. Finally, puromycin acts as a competitive inhibitor of the binding of aminoacyl-tRNA's to the so-called aminoacyl site (i.e., the A-site) of the ribosome and acts as a chain-terminator of the elongation phase as a result of its incorporation into the growing peptide chain.

Shortcomings with previously available assays for peptidyl transferase activity have hampered the search for novel modulators of peptidyl transferase activity. For example, many previously available assays require the use of radioactive compounds, suffer from a lack of sensitivity, or both. Moreover, previously available assays for peptidyl transferase activity are not amenable to high throughput screening methods such as are needed to screen large libraries or groups of potential modulators. Thus, there remains a need in the art for new assay methods for identifying modulators of peptidyl transferase activity. The present invention remedies this and other needs.

### SUMMARY

In one aspect, the present invention features novel synthetic methods for the preparation of CpCpA-3′-deoxy-3′-N-phenylalanine (Compound I), a valuable tool for investigations of the mechanism of enzymatic activity of ³²pCpCpA-NH-Phe.

The present invention also includes synthetic procedures for the preparation of species related to Compound I, e.g., pCpCpA-O-Met-Biotin, pCpCpA-O-Phe-Phe-Met-Biotin, tRNA-O-Met-Biotin, and pCpCpA-NH-Phe. These substrates are highly reactive for the peptidyl transferase (PT) reaction in the 70S and 50S large subunits. With the crystal structure of the ribosome now available, insight into RNA-mediated peptidyl transferase activity is likely to have broad implications. Thus, the compounds of the present invention provide additional utility in generating information essential to elucidation of the mechanisms of protein synthesis.

In one aspect, the invention provides compounds of formula (I),

X-CpCpA-NH-Phe (I)

wherein X is absent or a label. The label can be, e.g., radioactive (e.g., ³²P) or fluorescent, and can be attached to the 5′-hydroxy group. In certain embodiments, the NH-Phe moiety can be replace with another moiety, such as Met-Biotin, 0-Met-biotin NH-Phe-Boc, or Phe-Met-Biotin.

In another aspect, the invention relates to methods of making the compounds delineated herein, including any one or more of the steps, reagents, intermediate compounds or sequence of reactions, as delineated in any of FIGS. 1-3 or any of the examples herein.

For example, the invention provides methods of making a compound of formula (I) that includes providing a compound of formula (II):

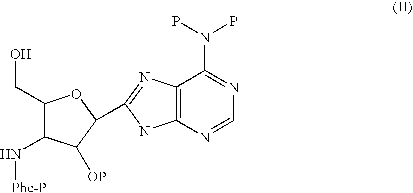

wherein each P is independently an oxygen- or nitrogen-protecting group; and converting it to a hydroxyl-group protected derivative of formula (III):

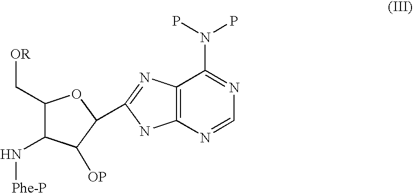

wherein each R is an oxygen-protecting group; and each P is independently an oxygen- or nitrogen-protecting group. In some embodiments, R can be a silyl group or tert-butyldiphenylsilyl or dimethoxytrityl. P can be benzyl.

In other embodiments, the methods can further include converting the hydroxyl-group protected derivative to a compound of formula (I). The methods can include removing the protecting group from the hydroxyl-group protected derivative and attaching a label to the resulting hydroxyl-group. The label (e.g., a radioactive (e.g., ³²P) label can be attached by reaction of γ-³²P-ATP with the resulting hydroxyl-group.

In another aspect, the invention provides methods for monitoring peptide bond formation. The methods include providing a mixture that includes a peptidyl transferase, a peptidyl-tRNA analog, and an aminoacyl-tRNA analog of formula (I), incubating the mixture under conditions sufficient to enable peptide bond formation, and monitoring the mixture for peptide bond formation. The aminoacyl-tRNA analog can be ³²p*CpCpA-NH-Phe. The mixture can further include a test compound, and the method can be used to monitor the effect of the test compound on peptide bond formation. The peptidyl-tRNA analog can be an amino acid conjugated to an oligonucleotide, e.g., 5′-CCA-phenylalanine, 5′-CACCA-phenylalanine, 5′-CACCA-methionine, 5′-CAACCA-formylmethionine, or tRNA-phenylalanine. The peptidyl transferase can be a ribosomal subunit. The aminoacyl-tRNA analog can be capable of being detected by polyacrylamide gel electrophoresis (PAGE).

The invention also provides methods for screening test compounds, e.g., to identify modulators (e.g., inhibitors) of peptidyl transferase activity. The methods include (a) incubating a mixture including a peptidyl transferase, a peptidyl-tRNA analog, an aminoacyl-tRNA analog of formula (I), and one or more test compounds; and (b) determining the rate of transfer of the peptidyl moiety of the peptidyl-tRNA analog to the free amino group of the aminoacyl-tRNA analog for each test compound. For example, a decrease in the rate can indicate that the compound is a modulator (e.g., inhibitor) of peptidyl transferase activity. The screening can be performed in a high-throughput format, and the test compounds can be members of a combinatorial library. The peptidyl transferase can be a ribosomal subunit.

Also provided are methods for determining whether a test compound is an inhibitor of peptidyl transferase activity, e.g., a candidate antibacterial or antifungal agent, or candidate inflammation or immune system modulating agent. The methods include (a) incubating a mixture including a peptidyl transferase, a peptidyl-tRNA analog, an aminoacyl-tRNA analog of formula (I), and one or more test compounds (e.g., under conditions allowing transfer of the peptidyl moiety of the peptidyl-tRNA analog to the aminoacyl-tRNA analog); (b) determining the rate of transfer of the peptidyl moiety of the peptidyl-tRNA analog to the free amino group of the aminoacyl-tRNA analog for each test compound; and (c) identifying one or more test compounds that inhibit peptidyl transferase activity. A test compound that inhibits peptidyl transferase activity is a peptidyl transferase activity inhibiting agent, e.g., a candidate antibacterial or antifungal agent, or a candidate inflammation or immune system modulating agent. The present invention also includes inhibitors of peptidyl transferase activity identified using any of the methods described above, and methods of inhibiting peptide synthesis using the compounds described herein. For example, a method of inhibiting peptide synthesis can include administering to a cell (e.g., in vivo or in vitro) an amount of a compound described herein effective to inhibit peptide synthesis in the cell.

The present invention also provide kits including a compound of formula (I) and instructions for using the compound in an assay to determine peptidyl transferase activity.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

FIG. 1 is a diagram illustrating the reaction scheme for synthesis of 2′-O-benzoyl-N,N-dibenzoyl-3′-deoxy-3′-N-(Boc-phenylalanyl) adenosine with 5′-TBDPS protection.

FIG. 2 is a diagram illustrating the reaction scheme for synthesis of 2′-O-benzoyl-N,N-dibenzoyl-3′-deoxy-3′-N-(Boc-phenylalanyl) adenosine with 5′-DMTr protection.

FIG. 3 is a diagram illustrating the reaction scheme for synthesis of cytidylyl(3′→5′phosphoryl)cytidylyl((3′→5′phosphoryl)-3′-deoxy-3′-(L-phenylalanyl) amido adenosine.

FIG. 4 is an autoradiogram of the peptidyl transferase reaction of 2′(3′)-biotin-methionyl-tRNA with 5′-³²pCpCpA-NH-Phe in the presence of E. coli 70S ribosome or S30 extract in the reaction buffer [20 mM Tris•HCl (pH 7.4), 6 μM Spermidine, 400 mM NH₄Cl, 4 mM MgCl₂, 330 mM KCl]. The samples were run on 7.5 M urea/20% polyacrylamide gel with 1×TBE buffer at 30 W. The bottom bands are ³²pCpCpA-NH-Phe and the top bands are ³²pCpCpA-NH-Phe-Met-biotin.

FIG. 5A-5C, in three panels, provides autoradiograms illustrating the presence of reaction products on polyacrylamide gel. FIG. 5A: substrates: ³²P-CCA-NH-Phe and CCA-Met-Biotin; reactions 1-3 (lanes 1-9): negative controls; lowest band-labeled ³²P-CCA-NH-Phe; middle band-formed dipeptide (³²P-CCA-NH-Phe-Met-Biotin), biotin-labeled; highest band-³²P-CCA-NH-Phe-Met-Biotin::streptavidin complex; FIG. 5B. illustrates effects on 70S-catalyzed peptidyl transferase activity in the absence of different components (spermidine, Tris•HCl, Mg²⁺, K⁺, or NH₄Cl); FIG. 5C illustrates the effects on peptidyl transferase activity as a function of different ribosomal components (S30 extract, 70S, 50S and 30S).

FIG. 6 is an autoradiogram illustrating products of peptidyl transferase reactions on polyacrylamide gels as a function of the presence of metal ions (Mg²⁺, Ca²⁺, Mn²⁺, Zn²⁺, Co²⁺, Cu²⁺) in 50S-catalyzed reactions.

FIGS. 7A-7C are line graphs illustrating the dependence of divalent metal ion concentration (FIG. 7A: Mg²⁺; FIG. 7B: Ca²⁺; FIG. 7C: Mn²⁺) on reaction kinetics of 50S-catalyzed reaction.

FIGS. 8A-8B are line graphs illustrating the effects on kinetic parameters as a function various concentrations of CCA-Met-Biotin in 50S A or S30 extract B.

FIGS. 9A-9C are graphs illustrating the effects of pH on the peptidyl transferase reaction. FIG. 9A: pH dependence in either 50S (●) or S30 extract (◯); FIG. 9B: pH dependence in 50S as a function of divalent metal ion; FIG. 9C: pH/pD dependence in 50S with 20 mM Mg²⁺.

FIGS. 10A-10B are autoradiograms of the peptide bond formation of pCpCpA-NH-Phe with pCpCpA-O-Met-Biotin or tRNA-O-Met-Biotine in E. coli S30 extract (FIG. 10A) and 70S ribsome (FIG. 10B).

FIG. 11 is a line graph illustrating the inhibition by exposure to antibiotics on peptide bond formation of pCpCpA-NH-Phe with pCpCpA-O-Met-Biotin in the 50S ribosome.

FIG. 12 is a line graph illustrating the inhibition by exposure to antibiotics on peptide bond formation of pCpCpA-NH-Phe with tRNA-O-Met-Biotin in the 50S ribosome.

FIG. 13 is a line graph illustrating the inhibition by exposure to antibiotics of peptide bond formation of pCpCpA-NH-Phe with pCpCpA-O-Met-Biotin in the 70S ribosome.

FIG. 14 is a line graph illustrating the inhibition by exposure to antibiotics of peptide bond formation of pCpCpA-NH-Phe with tRNA-O-Met-Biotin in 70S ribosome.

### DETAILED DESCRIPTION

The present invention provides methods for the direct monitoring of peptide bond formation in the ribosome. High throughput methods, compositions, kits, and integrated systems are provided for detecting peptidyl transferase activity. Accordingly, the present invention has at least two distinct utilities. First, methods for the preparation of CpCpA-3′-NH-phenylalanine as a ribosomal substrate is provided. The method employs simple reagents that can be readily synthesized and obtained in sufficient quantities. Second, the invention provides methods for screening new antibiotic drugs targeted to the ribosome with the added advantage of displaying higher sensitivity in monitoring peptidyl transferase activity. Moreover, the system does not require the addition of alcohol to the reaction mixture. Accordingly, the assays of the present invention are valuable for the identification of lead compounds for drug design, particularly for the development of antibacterial, antifungal, inflammation modulatory, or immune system modulatory agents. As such, the present invention is well suited for high throughput screening of active drug candidates. Further, the compounds described herein are valuable for inhibiting peptide synthesis, e.g., by administering to a cell (e.g., in vivo or in vitro) an amount of a compound described herein effective to inhibit peptide synthesis in the cell.

In fragment reactions of the ribosome, CAACCA-(fMet), CCA-(fMet), or even CA-fMet, can replace the P-site tRNA, while puromycin serves as an aminoacyl acceptor. When puromycin is used as the acceptor, the addition of alcohol (10-30%) is required. The nucleotides beyond the CCA sequence do not play an important role in binding to the A-site, as indicated by the similar binding activities of CCA-Phe and CACCA-Phe. Therefore, only nucleotides of the CCA terminus play an important role in the binding of aminoacyl-tRNA to the A-site. In this regard, it has been reported that tRNA bearing 3′-amino-3′-deoxyadenosine in the final sequence retained full acceptor activity.

Investigation of peptidyltransferase activity has been hampered by technical problems associated with the complexity of the ribosome and its substrates. Most studies in peptidyltransferase have largely utilized two methods: fragment reaction (Monro, R. E.; Marcker, K. A. J. Mol. Biol. 1967, 25, 347-350); and poly (Phe) synthesis (Fahnestock, S.; et al., Methods Enzymol. 30: 554-562 (1974)). In the fragment assay, peptidyltransferase activity is measured by formation of N-formyl-[35S]Met-puromycin from a fragment of N-formyl-Met-tRNAfMet (as the peptidyl donor) and puromycin (as the peptidyl acceptor). Puromycin was initially presumed to be an inhibitor that competitively binds to the A-site in the ribosome and prematurely to the C terminus of the peptide. However, recent studies revealed multiple modes of puromycin inhibition. Starck, S. R. and Roberts, R. W., RNA 8: 890-903 (2002); Miyamoto-Sato, E., et al., Nucleic Acids Research 28: 1176-1182 (2000). In the poly (Phe) synthesis, the peptidyltransferase activity is reflected by the release of synthesized polyPhe peptide. The formation of peptide bonds is not directly observed. Another disadvantage of these assays is the tedious analysis of the activity. Reaction products are analyzed either by high-voltage paper electrophoresis and phosphorimage quantitation, or by estimating the radioactivity in the ethyl acetate-extracted fraction by scintillation counting.

To overcome these obstacles and to gain a better understanding of the mechanistic aspects of ribosomal peptidyltransferase, the present inventors have developed a simple system with novel substrates (CCA-NH-Phe and CCA-Met-Biotin) for characterizing ribosomal peptidyl transferase activity. Conventional activity assays employ a fragment of tRNA and puromycin in testing ribosome activity. The compounds of the invention, e.g., CCA-NH-Phe, are the first designed A-site substrates to replace either puromycin, C-puromycin, or CC-puromycin, and they more closely mimics the natural ribosomal substrate. With these substrates, dipeptide products are quickly formed in the presence of ribosome components and products are readily resolved by high-percentage polyacrylamide gel.

Thus, the pCpCpA-NH-Phe of the present invention exhibits full activity for the peptidyl transferase reaction in the ribosome. After radiolabeling with ³²P at the 5′-end of CpCpA-NH-Phe, the peptidyl transferase (PT) reaction can be monitored by polyacrylamide gel electrophoresis (PAGE) with high sensitivity. and analyzed quantitatively with, for example, a Molecular Dynamics PhosphorImager™.

The trinucleotide cytidylyl(3′→5′ phosphoryl)cytidylyl(3′→5′ phosphoryl)-3′-deoxy-3′-(L-phenylalanyl) amido adenosine (CpCpA-NH-Phe) was synthesized by phosphoramidite chemistry from 3′-amino-3′ deoxyadenosine as the ribosomal substrate. The 3′-amino-3′-deoxyadenosine was first converted to 3′-(N-tert-butyloxycarbonyl-L-phenylalanine)amido-3′-deoxy-6-N,6-N,2′-O-tribenzoyl-adenosine and then coupled with cytidine phosphoramidite to produce the fully protected CpCpA-NH-Phe-Boc. The product was obtained after removing all protection groups and then radio-labeled with 32P to yield p*CpCpA-NH-Phe, that demonstrated high activity for the peptidyl transferase reaction in the ribosome.

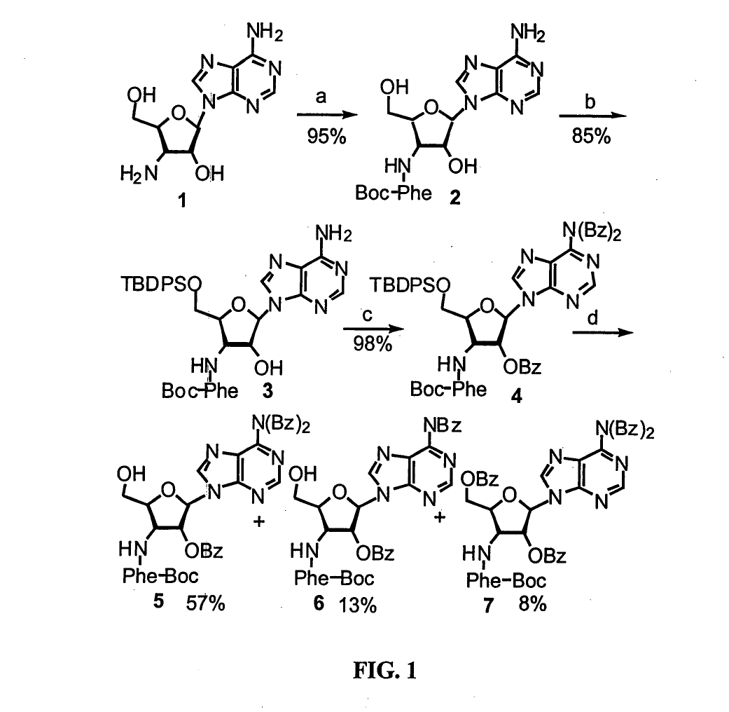

In reference to FIGS. 1-3, the target compound was synthesized from 3′-amino-3′-deoxy-adenosine Compound 1 by phosphoramidite chemistry. Robins's nine-step route for the synthesis of 3′-amino-3′-deoxyadenosine Compound 1 has been modified into a seven-step process with 55% overall yield. Briefly, adenosine was protected with tert-butyldiphenylsilyl at the 5′-position and then treated with α-acetoxyisobutyryl bromide to yield 2′-O-acetyl-3′-bromo-3′-deoxy-5′-O-tert-butyldiphenylsilyl-adenosine. This was treated with ammonia in methanol and then reacted with benzylisocyanate to yield 3′-(benzylamino)-5′-O-(tert-butyl)diphenylsilyl-3′-N,2′-O-carbonyl-3′-deoxyadenosine. This product was treated with sodium hydride, then with base, and was finally deprotected to yield 3′-deoxy-3′-amino-3′-deoxyadenosine Compound 1. Since Compound 1 contains multiple functional groups, finding a suitable protection group for each functional group is critical toward the synthesis of CpCpA-NH-Phe.

Boc-L-Phenylalanine was first introduced into the 3′-position of 3′-amino-3′-deoxyadenosine Compound 1, which also acted as a protection group of the 3′-amino group. The reaction in DMF was not successful because of the poor solubility of Compound 2 in DMF and a high racemization of L-phenylalanine. When N-(tert-butyloxycarbonyl)-L-phenylalanine N-hydroxy succinimide ester was stirred with 3′-amino-3′-deoxyadenosine Compound 1 in anhydrous dimethyl sulfoxide (DMSO), an optically pure 3′-(N-tert-butyloxycarbonyl-L-phenylalanine)amido-3′-deoxyadenosine Compound 2 was obtained. The tert-butyl-diphenylsilyl (TBDPS) group was first used to protect the 5′-hydroxyl group of Compound 2 to yield 5′-O-(tert-butyl-diphenylsilyl)-3′-(N-tert-butyloxycarbonyl-L-phenylalanine) amido-3′-deoxy-adenosine Compound 3 (FIG. 1). This reaction was highly regioselective to form the 5′-protected compound. After benzoylation, the fully protected 3′-amino-3′-deoxyadenosine (98%) was treated with a fluoride agent to remove the 5′-O-TBDPS group. The desired Compound 5 was thus produced, along with mono- and tetra-benzoyl analogs.

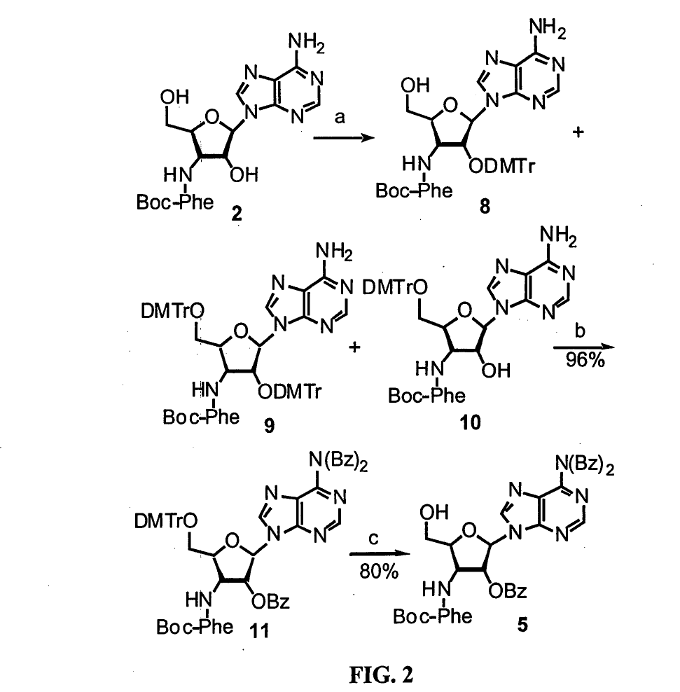

The present inventors have investigated the DMTr protection of the 5′-hydroxy of Compound 2. Reaction of Compound 2 with 4,4′-dimethoxyltrityl chloride in pyridine yielded a mixture of components (FIG. 2). The mixture was separated on a flash column of silica gel to give the desired product 3′-(N-tert-butyloxycarbonyl-L-phenylalanine)amido-3′-deoxy-5′-O-(4,4′-dimethoxytrityl)-adenosine Compound 10, unreacted Compound 2, 3′-(N-tert-butyloxycarbonyl-L-phenylalanine) amido-3′-deoxy-2′-O-(4,4′-dimethoxytrityl)-adenosine Compound 8, and 2′-O,5′-O-bis(4,4′-dimethoxytrityl)-3′-(N-tert-butyloxycarbonyl-L-phenylalanine)amido-3′-deoxyadenosine Compound 9. Compounds 8 and 9 were treated with acetic acid to regenerate the starting material Compound 2 without the cleavage of the N-Boc group of phenylalanine. This allowed for efficient recovery of starting material.

Benzoylation of Compound 10 with a large excess amount of benzoyl chloride produced 3′-(N-tert-butyloxycarbonyl-L-phenylalanine) amido-3′-deoxy-5′-O-(4,4′-dimethoxytrityl)-6-N,6-N,2′-O-tribenzoyl-adenosine Compound 11. Deprotection of the DMTr group with acetic acid afforded the desired Compound 5.

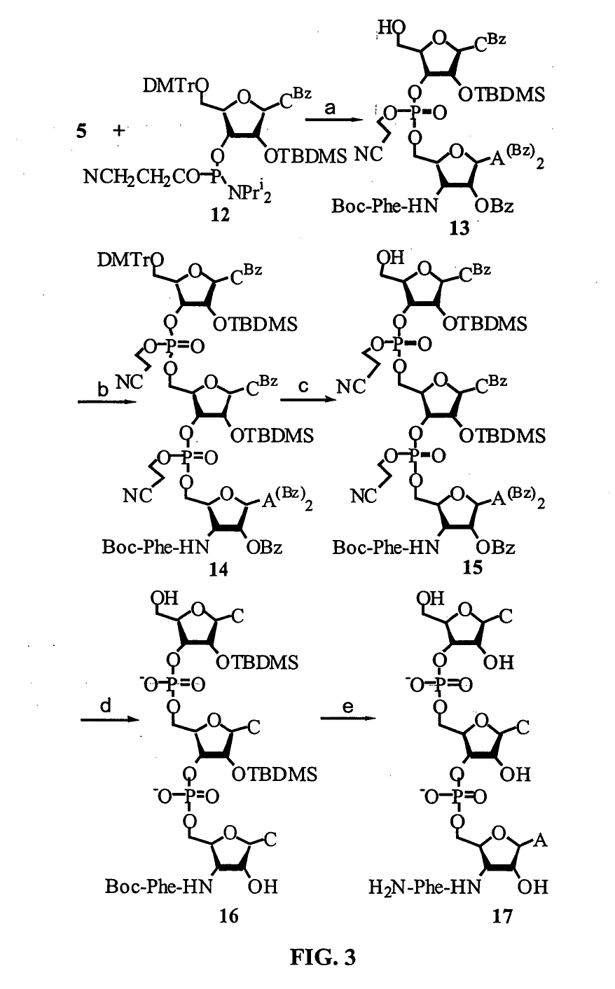

The synthesis of 5′-HO-CpCpA-3′-N-Phe Compound 17 depicted in FIG. 3 was based upon a similar strategy of phosphoramidite methodology. The 5′-hydroxy group of Compound 5 was coupled with a commercially available cytidine phosphoramidite Compound 12 in the presence of 1H-tetrazole in anhydrous acetonitrile, oxidized by t-butyl hydroperoxide, and then treated with acetic acid to yield Compound 13. Dinucleotide Compound 13 was coupled with Compound 12 again to yield the fully protected trinucleotide Compound 14. After purification, Compound 14 was treated with acid to produce Compound 15. Deprotection of benzoyl and cyanoethyl groups afforded trinucleotide Compound 16. The N-Boc group of Compound 16 was subsequently removed and the resulting compound was purified to give Compound I as a white solid.

Protecting groups and protecting group methodologies (protection and deprotection) useful in synthesizing applicable compounds herein are known in the art and include, for example, those described in T. W. Greene and P. G. M. Wuts, Protective Groups in Organic Synthesis, 3rd Ed., John Wiley and Sons (1999), and subsequent editions thereof.

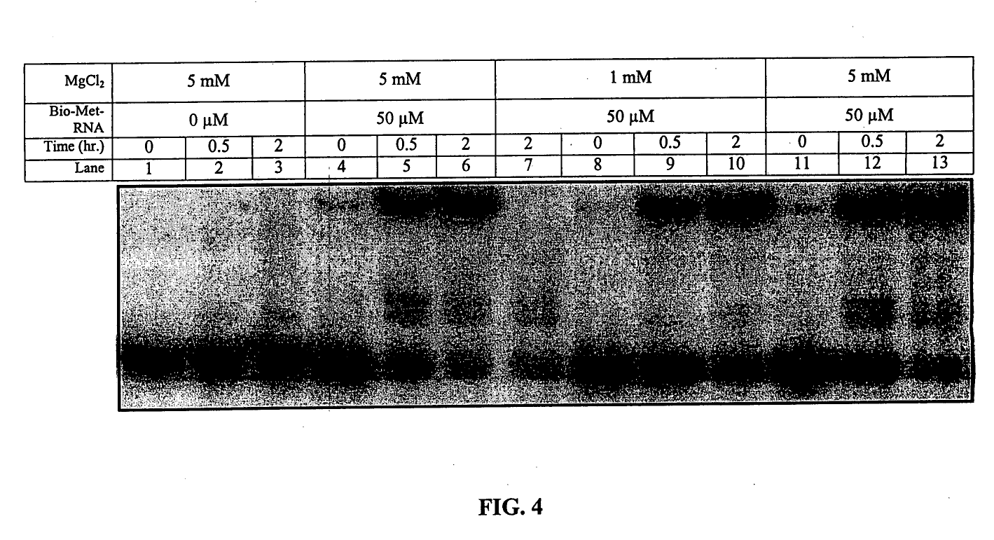

The following is intended to demonstrate, but not limit, the application of this invention. Labeling the 5′-end of trinucleotide 17 was accomplished through phosphorylation using T4 polynucleotide kinase (PNK) and [γ-³²P]ATP. It can also be done by additional methods known to those skilled in the art. The 5′-end labeled ³²pCpCpA-NH-Phe was used as a peptidyl acceptor for the peptidyl transferase reactions in the ribosome. These reactions were monitored by polyacrylamide gel electrophoresis (PAGE) (FIG. 4). Other methods of monitoring commonly used for such labels can also be used. No product was formed in absence of tRNA-O-Met-biotin substrate in S30 extract (Promega) with p*CpCpA-NH-Phe (lanes 1-3, FIG. 4). When p*CpCpA-NH-Phe was incubated with 50 μM tRNA-O-Met-biotin in the presence of 1.0 U E. coli S30 extract (lanes 4-7, FIG. 4) under the reaction buffer, a new band was formed.

When p*CpCpA-NH-Phe was incubated with 50 μM tRNA-O-Met-biotin in the presence of 1.0 U E. coli 70S ribosome under the same reaction buffer, a same product band was formed at an approximate 90% yield at the reaction's final extent (lanes 8-13, FIG. 4). After treatment with streptavidin, the product band migrated much more slowly (the band not shown in the figure, lane 7) indicating that a biotin moiety was transferred to *pCpCpA-NH-Phe. This suggested that a peptide bond was formed between pCpCpA-NH-Phe and tRNA-O-Met-biotin. These results demonstrated that pCpCpA-NH-Phe is fully active for the peptidyl transferase reaction in the ribosome.

Six antibiotics (puromycin, chloramphenicol, sparsomycin, tetracycline, streptomycin, and erythromycin) have been used as inhibitors for the peptidyl transferase reaction in the ribosome with the chemically synthesized substrates. Puromycin is a structural mimic of the 3′-end nucleotide of Tyr-tRNATry. Puromycin can enter the ribosomal A-site and act as an acceptor substrate. Chloramphenicol is a direct inhibitor of the peptidyl bond formation. Sparsomycin is a broad spectrum inhibitor that competes with puromycin for ribosome binding and blocks the binding of tRNAs to the A-site. Erythromycin may block the entrance to the tunnel through which the growing peptide chain leaves the ribosome. Tetracycline demonstrates strong binding to the 30S subunit and streptomycin leads mRNA misreading. See Table 1.

TABLE 1
ANTIBIOTICS AND THEIR INTERACTIONS
WITH THE RIBOSOME

| Antibiotic | Interaction |
|---|---|
| Sparsomycin | P-site binding inhibition on 50 S |
| Erythromycin | P-site binding inhibition and exit tunnel |
| | inhibition |
| Streptomycin | mRNA misreading |
| Tetracycline | A-site binding inhibition on 30 S |
| Chloramphenicol | A-site binding inhibition on 50 S |
| Puromycin | A-site binding inhibition and premature |
| | linkage to the C terminus of synthesized |
| | peptides |

Peptidyltransferase activity was tested with various kinds of antibiotic inhibitors in the presence of different ribosomal components.

Assay reactions were carried out with: 50S or 70S subunits from the bacterial ribosome (prepared from E. coli MRE600), or S30 extract (purchased from Promega); 50 μM CCA-Met-Biotin or tRNA-Met-Biotin; trace amounts of ³²P-CCA-NH-Phe (˜0.5 nM); 100 μM antibiotic in the presence of 50 mM Tris•HCl (pH 7.5); 35 mM MgCl₂; 100 mM NH₄Cl; and 1000 mM KCl, at 37° C. For each reaction, the same amount of 50S or 70S (10 pmol) was included, but the S30 concentration was not determined.

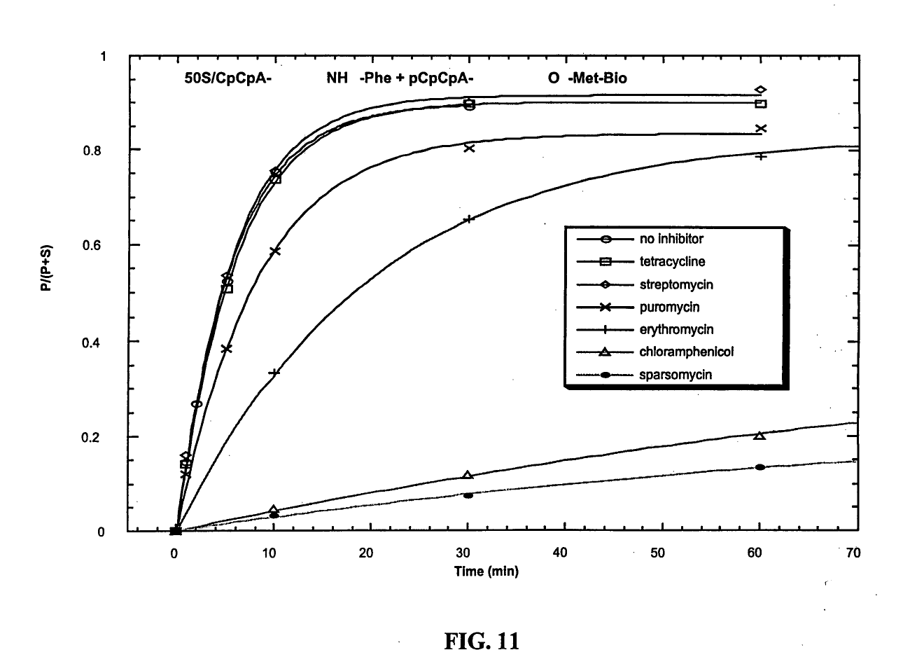

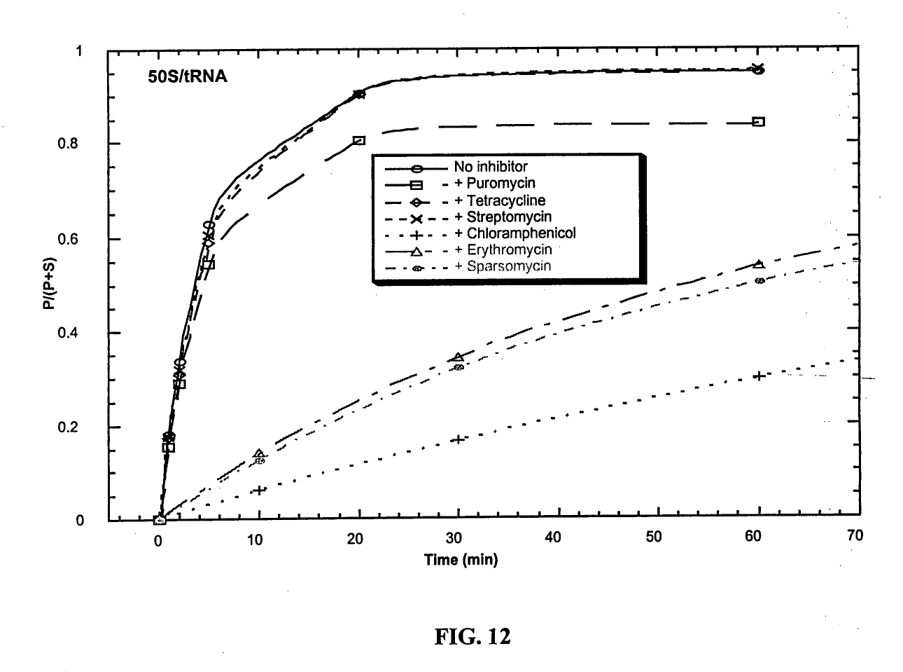

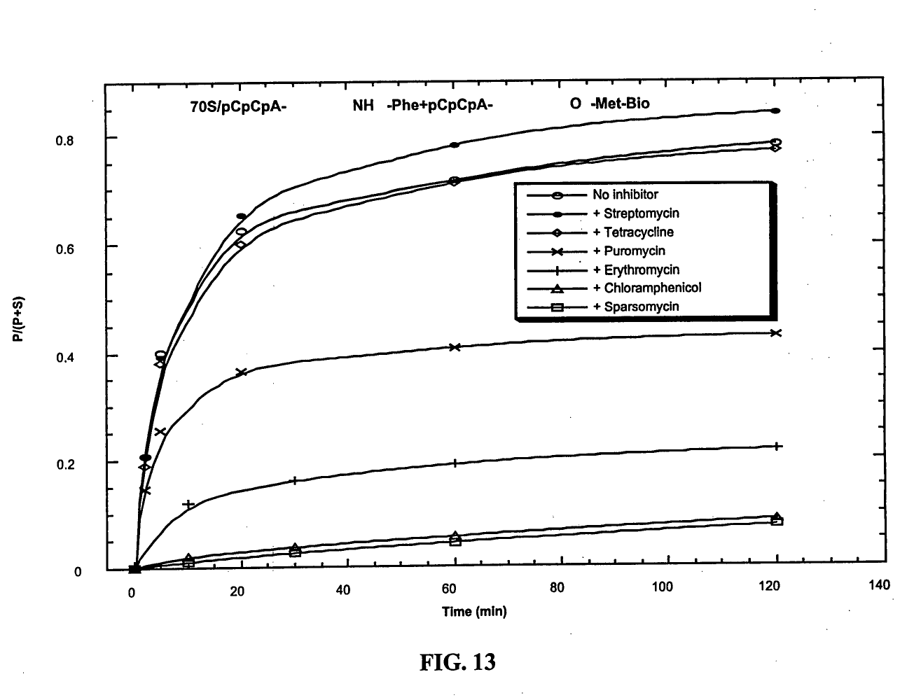

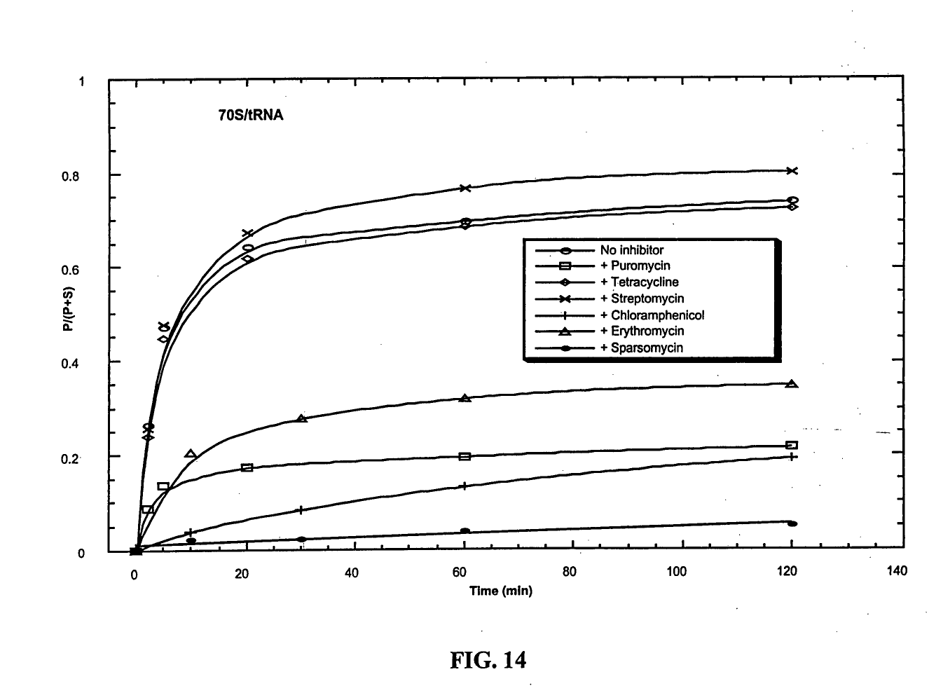

Reactions were initiated by addition of ³²P-CCA-NH-Phe. A 1-μL aliquot of the reaction mixture was removed at specific time points, quenched with 2 μL quench buffer (formamide +0.05% xylene cyanol), and loaded on 24%/7.5 M urea polyacrylamide denaturing gel. Reaction products were quantitated with a Molecular Dynamics PhosphorImager™, and the fraction of individual product relative to total reactants was plotted against time (see FIGS. 11-14).

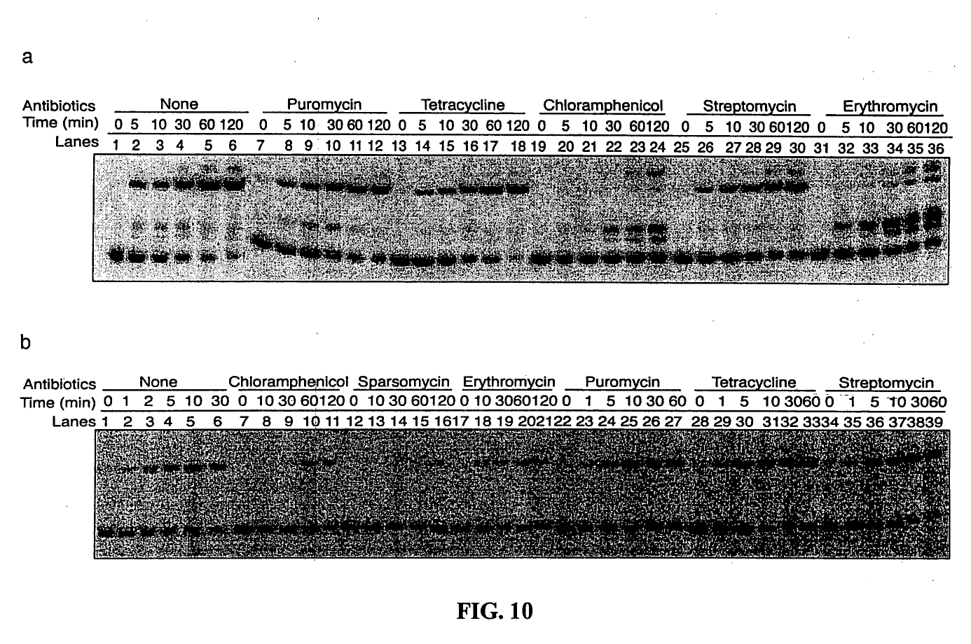

Provided are gel-shift assays of peptidyltransferase activity of S30 extract (FIG. 10A) and 70S ribosome (FIG. 10B) in the presence of different antibiotics. The substrates used in these reactions were CCA-Met-Biotin and ³²P-CCA-NH-Phe. The lower band corresponds to the labeled ³²P-CCA-NH-Phe and the top band is the dipeptide product (³²P-CCA-NH-Phe-Met-Biotin). In the absence of any antibiotics, the peptide bond is formed between the two substrates in S30 extract (lanes 1-6, FIG. 10A) and 70S ribosomes (lanes 1-6, FIG. 10B). Peptidyltransferase activity was not inhibited by either tetracycline or streptomycin (lanes 13-18 and 25-30, FIG. 10A; lanes 28-33 and 34-39, FIG. 10B). It is recognized that these compounds do not inhibit peptide bond formation, which is believed to occur on the 50S subunit.

Peptidyltransferase activity was greatly inhibited by chloramphenicol, erythromycin and sparsomycin (lanes 19-24 and 31-36, FIG. 10A; lanes 7-11, 12-16 and 17-21, FIG. 10B), which agrees with previous findings that these antibiotics directly inhibit peptide bond formation in the peptidyltransferase center of the ribosome. However, 100 μM puromycin does not significantly inhibit peptidyltransferase activity in S30 extract or 70S ribosome (lanes 7-12, FIG. 10A; lanes 22-27, FIG. 10B). Puromycin is thought to inhibit protein synthesis via either competitive binding to the A-site, or premature linking to the C-terminus of synthesized peptides. Recently, it has been suggested that puromycin may affect ribosome activity through multiple modes of interaction. The results disclosed herein reveal that 100 μM puromycin does not show strong inhibition of peptide bond formation when CCA-Met-Biotin and ³²P-CCA-NH-Phe was used as the ribosomal substrate, suggesting that pCpCpA-NH-Phe has a much stronger binding affinity than puromycin in the active site of protein synthesis.

The results of inhibition studies of peptidyltransferase activity of ³²pCpCpA-NH-Phe in 70S or 50S ribosome with either pCpCpA-Met-Biotin or tRNA-Met-Biotin substrates in the presence of different antibiotics are provided in FIGS. 11-14. Reaction conditions for these studies were the same as described above. In either 70S- or 50S-mediated peptidyl transferase reactions with either CCA-Met-Biotin or tRNA-Met-Biotin substrates, tetracycline and streptomycin serve as positive controls that show almost identical activity as in the absence of an inhibitor. Other antibiotics (puromycin, sparsomycin, erythromycin and chloramphenicol) demonstrated different ability for the inhibition of peptidyl transferase activity. Chloramphenicol showed strong inhibition for both 70S- and 50S-mediated reactions, and the activity difference between CCA-Met-Biotin and tRNA-Met-Biotin were similar in 70S and 50S. Sparsomycin strongly inhibited reactions in 70S; however, the activity of 50S-tRNA was much less unaffected than that of 50S-CCA. Both Erythromycin and puromycin inhibited 70S reactions much more strongly than 50S reactions. Comparison of peptidyl transferase activity between 70S and 50S, CCA-Met-Biotin, and tRNA-Biotin with different antibiotics provides useful information on how the antibiotics interact with the ribosome.

The results of antibiotic inhibition studies with pCpCpA-NH-Phe and pCpCpA-O-Met-Biotin agree well with biochemical and crystal results concerning the antibiotic binding site in the ribosome. The results disclosed herein further confirmed these antibiotic binding sites. The synthetic substrates of the present invention also demonstrated utility in screening assays of new antibiotic agents that target on the ribosome.

The present invention also provides solution or solid phase assays for measuring the activity of peptidyl transferase in the presence of a peptidyl transferase activity modulator. High throughput methods, compositions, kits and integrated systems are provided for detecting peptidyl transferase activity in vitro and for measuring the effect of potential modulators of peptidyl transferase activity. Accordingly, the assays of the present invention have, inter alia, at least two proven utilities. First, the assays can be used to detect peptidyl transferase activity in vitro, serving as a replacement for standard cell-free assays that target protein synthesis in general. As such, the assays provide broadly applicable tools for assessing peptidyl transferase activity in a high-throughput format.

Second, the assays provide for the identification of inhibitors of peptidyl transferase activity. Such modulators are valuable research tools that can be used to elucidate the biochemistry and enzymology of protein synthesis in both prokaryotic and eukaryotic systems. Moreover, such modulators provide lead compounds for drug development to treat a variety of conditions, including the development of antibacterial, antifungal, inflammation, or immune system modulatory agents. Accordingly, the assays of the present invention are of immediate value as a result of their ability to identify lead compounds for pharmaceutical and/or other research applications. Moreover, such assays are particularly well suited to high throughput automation, making them particularly valuable for their ability to identify lead compounds.

In the assays of the present invention, either intact ribosomes (i.e., the 70S ribosome for prokaryotes or the 80S ribosome for eukaryotes) or, alternatively, the ribosomal subunits to which the peptidyl transferase has been localized (i.e., the 50S subunit for prokaryotes or the 60S for eukaryotes) can be employed. The ribosome subunits need not be complete, as long as the components required for peptidyl transferase activity are present.

Intact ribosomes or, alternatively, the ribosomal subunit of interest, can be isolated using standard methods and procedures known to those of skill in the art. For instance, ribosomes and supernatant factors from E. coli can be prepared according to standard procedures as described by Nishizuka, Y, & Lipmann, F., Proc. Nat'l, Acad. Sci., Wash., 55: 212 (1967); Stachelin and Maglott, Meth. Enzym. 20: 449 (1971); and Sanchez-Madrid, et al., Eur. J. Biochm. 98: 409 (1979). Typically, ribosomes extracted from micro-organisms are prepared according to the following successive stages: (1) culture of the microorganism strain on a liquid or solid medium; (2) collection and lysis of the microorganism; and (3) treatment of the lysate by repeated centrifugation at progressively higher speeds to obtain the fraction containing the ribosomes, which fraction can then be further purified. Moreover, methods for the preparation of ribosomes from eukaryotic cells vary somewhat depending on the source organism. However, procedures that can be used to isolate ribosomes derived from eukaryotic cells and that are suitable for scale-up to the volume required for utilization in the high-throughput screening assays of the present invention are known to those of skill in the art. For example, procedures for isolating 80S ribosomes from rabbit reticulocytes are described by Allen, E. H. and Schweet, R. S., J. Biol. Chem., 237:760-767 (1962); and loannou, M., et al., Anal. Biochem., 247:115-122 (1997).

The invention also provides methods of identifying compounds that modulate peptidyl transferase-activity. Essentially any chemical compound can be used as a potential activity modulator in the assays of the invention, although most often compounds that can be dissolved in aqueous or organic (especially DMSO-based) solutions are used. The assays are designed to screen large chemical libraries by automating the assay steps and providing compounds from any convenient source to assay, which are typically run in parallel (e.g., in microliter formats on microliter plates in robotic assays). It will be appreciated by those of skill in the art that there are many commercial suppliers of chemical compounds, including Sigma Chemical Co. (St. Louis, Mo.), Aldrich Chemical Co. (St. Louis, Mo.), Sigma-Aldrich (St, Louis, Mo.), Fluka Chemika-Biochemica Analytika (Buchs, Switzerland), and the like.

In one preferred embodiment, high throughput screening methods involve providing a combinatorial library containing a large number of potential therapeutic compounds (potential modulator compounds). Such “combinatorial chemical libraries” are then screened in one or more assays to identify those library members (particular chemical species or subclasses) that display a desired characteristic activity. The compounds thus identified can serve as conventional “lead compounds,” or can themselves be used as potential or actual therapeutics.

A combinatorial or other chemical or compound library is a collection of diverse test compounds generated by either chemical synthesis or biological synthesis, by combining a number of chemical “building blocks,” such as reagents. For example, a linear combinatorial chemical library, such as a polypeptide library, is formed by combining a set of chemical building blocks (amino acids) in every possible way for a given compound length (i.e., the number of amino acids in a polypeptide compound). Millions of chemical compounds can be synthesized through such combinatorial mixing of chemical building blocks. The test compounds can be small organic or inorganinc molecules, oligonucleotides, peptides, polypeptides, polysaccharides, and the like.

Preparation and screening of compound libraries, such as combinatorial chemical libraries, is well known to those of skill in the art. Such combinatorial chemical libraries include, but are not limited to, peptide libraries (see, e.g., U.S. Pat. No. 5,010,175; Furka, Int. J. Pept. Prot. Res., 37: 487-493 (1991); and Houghton, et al., Nature, 354:84-88 (1991)). Other chemistries for generating chemical diversity libraries can be used. Such chemistries include, but are not limited to, peptides (PCT Publication No. WO 91/19735); encoded peptides (PCT Publication WO 93/20242); random biooligomers (PCT Publication No. WO 00091); benzodiazepines (U.S. Pat. No. 5,288,514); diversomers, such as hydantoins, benodiazepines and dipeptides (Hobbs, et al., Proc. Nat. Acad. Sci. USA 90: 6909-6913 (1993)), and many others.

Devices for the preparation of combinatorial libraries are commercially available (see, e.g., 357 MPS, 390 MPS, Advanced Chem. Tech, Louisville, Ky.; Symphony, Rainin, Woburn, Mass.; 433A Applied Biosystems, Foster City, Calif., 9050 Plus, Millipore, Bedford, Mass.). In addition, numerous combinatorial libraries are themselves commercially available (see, e.g., ComGerarx, Princeton, N.J., Asinex, Moscow; Tripos, Inc., St. Louis, Mo.; ChemStar, Ltd., Moscow; 3D Pharmaceuticals, Exton, Pa.; Marck Biosciences, Columbia, Md., etc.)

As noted, the invention provides in vitro assays for peptidyl transferase activity in a high-throughput format. Control reactions that measure peptidyl transferase activity in a reaction that does not include a peptidyl transferase activity modulator are optional, as the assays are highly uniform. Such optional control reactions are appropriate and increase the reliability of the assay. Accordingly, in some embodiments, the methods of the invention include such a control reaction. For each of the assay formats described, “no modulator” control reactions, which do not include a modulator, provide a background level of binding activity.

The invention also provides integrated systems for high throughput screening of potential modulators for peptidyl transferase activity. Such systems typically include a robotic armature that transfers fluid from a source to a destination; a controller that controls the robotic armature; a label detector; a data storage unit that records label detection; and an assay component such as a microliter dish comprising a well having a capture moiety for a peptidyl-tRNA analog affixed to the well.

A number of well-known robotic systems have been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) that mimic the manual synthetic operations performed by a chemist. Any of the above devices are suitable for use with the present invention. The nature and implementation of modifications to these devices (if any) so that they can operate as discussed herein will be apparent to persons skilled in the relevant art.

Any of the assays for compounds that modulate peptidyl transferase activity, as described herein, are amenable to high throughput screening. High throughput screening systems are commercially available (see, e.g., Zymark Corp., Hopkinton, Mass.; Air Technical Industries, Mentor, Ohio; Beckman Instruments, Inc., Fullerton, Calif.; Precision Systems, Inc., Natick, Mass., etc.). Such systems typically automate entire procedures including all sample and reagent pipetting, liquid dispensing, timed incubations, and final readings of the microplate in detector(s) appropriate for the assay. These configurable systems provide high throughput and rapid start-up as well as a high degree of flexibility and customization. The manufacturers of such systems provide detailed protocols for the various high-throughput systems.

Optical images viewed (and, optionally, recorded) by a camera or other recording device (e.g., a photodiode and data storage device) are optionally further processed in any of the embodiments described herein, e.g., by digitizing the image and storing and analyzing the image on a computer. A variety of commercially available peripheral equipment and software is available for digitizing, storing and analyzing a digitized video or digitized optical image, e.g., using PC (Intel x86 or Pentium chip-compatible DOS™, OS2™, WINDOWS™, WINDOWS NT™, or WINDOWS 95, 98, etc.-based machines), MACINTOSH-, or UNIX-based (e.g., SUN™ work station) computers.

One conventional system carries light from the specimen field to a cooled charge-coupled device (CCD) camera, in common use in the art. A CCD camera includes an array of picture elements (pixels). The light from the specimen is imaged on the CCD. Particular pixels corresponding to regions of the specimen (e.g., individual hybridization sites on an array of biological polymers) are sampled to obtain light intensity readings for each position. Multiple pixels are processed in parallel to increase speed. The apparatus and methods of the invention are easily used for viewing any sample, e.g., by fluorescent or dark field microscopic techniques.

In the assays of the present invention, a reaction mixture comprising a peptidyl transferase, a peptidyl-tRNA analog, and an aminoacyl-tRNA analog, such as ³²p*CpCpA-NH-Phe, are incubated under conditions suitable for transfer of the peptidyl moiety of the peptidyl-tRNA analog to the aminoacyl-tRNA analog. The peptidyl-tRNA can comprise a peptidyl moiety and an immobilizable tag, whereas the aminoacyl-tRNA analog is capable of being detected by polyacrylamide gel electrophoresis or other highly sensitive, analytical techniques common in the art. In a presently preferred embodiment, the reaction mixture further comprises a potential modulator of peptidyl transferase activity. If peptidyl transferase activity is present and uninhibited, it catalyzes the transfer of the peptidyl moiety of the peptidyl-tRNA analog to the free amino group of the aminoacyl-tRNA analog.

In the assays of the present invention, the peptidyl-tRNA analog is any compound that can become associated with the peptidyl site, i.e., P-site, of a peptidyl transferase. For example, the peptidyl-tRNA analog is an amino acid conjugated to an oligonucleotide. Any amino acid can be conjugated to the oligonucleotide. It will be readily apparent to those skilled in the art that amino acids (both natural and synthetic) can be used in the assays of the present invention. Moreover, additional moieties (e.g., a label) can be added to the amino acid or oligonucleotide. The oligonucleotide to which the amino acid is conjugated can be a full length tRNA or a portion thereof. The amino acid can be conjugated to the oligonucleotide using standard conjugation methods known to and used by those of skill in the art. Preferred peptidyl-tRNA analogs for use in the assay methods of the present invention include, but are not limited to, 5′-CCA-phenylalanine, 5′-CACCA-phenylalanine, 5′-CAACCA-methionine, 5′-CAACCA-formylmethionine, and tRNA-phenylalanine.

In the assays of the present invention, one of the aminoacyl-tRNA analogs is p*CpCpA-NH-Phe, which can react with a peptidyl-tRNA analog to yield a peptidyl-aminoacyl-tRNA analog. Other species such as 5′-fluorescence-pCpCpA-NH-Phe, and 5′-fluorescence-tRNA-NH-Phe can also be used. These species are particularly useful in that pCpCpA-NH-Phe can be attached to any tRNA (minus CCA) by T4 RNA ligase. Moreover, as described herein, the aminoacyl-tRNA analog is capable of being detected directly (e.g., through the use of a label) or indirectly (e.g., through the use of a detection moiety that contains a label and can bind to the aminoacyl-tRNA analog).

A wide variety of labels can be used, with the choice of label depending on sensitivity required, ease of conjugation with the aminoacyl-tRNA analog, stability requirements, and available instrumentation and disposal provisions. Methods would be known to those skilled in the art.

In addition, the invention provides compositions, kits and integrated systems for practicing the assays described herein. For example, an assay composition having a peptidyl-tRNA analog, an aminoacyl-tRNA analog, an immobilizable tag bound to the peptidyl-tRNA analog, and a label bound to the aminoacyl-tRNA analog is provided by the present invention. Additional assay components as described herein are also provided. For instance, a solid support or substrate to which the tagged peptidyl-tRNA analog can be bound can also be included. Such solid supports include membranes (e.g., nitrocellulose or nylon), a microliter dish (e.g., PVC, polypropylene, or polystyrene), a test tube (glass or plastic), a dipstick (e.g., glass, PVC, polypropylene, polystyrene, latex, and the like), a microcentrifuge tube, or a glass, silica, plastic, metallic or polymer bead or other substrate such as paper. Most commonly, the assay will use 96-, 384-, or 1536-well microliter plates.

The invention also provides kits for practicing the peptidyl transferase screening assays described herein. The kits can include any of the compositions noted above, and optionally further include additional components such as instructions to practice a high-throughput method of screening for a peptidyl transferase activity modulator, one or more containers or compartments (e.g., to hold peptidyl-tRNA analogs, aminoacyl-tRNA analogs, modulators, or the like), a control activity modulator, a robotic armature for mixing kit components, and the like.

The invention will be further described in the following examples, which do not limit the scope of the invention defined by the claims.

### EXAMPLES

### Example 1

### Synthesis of CpCpA-NH-Phe

General reagents and materials. Solvents were dried using standard methods and distilled before use; TLC: precoated silica gel thin layer sheets 60 F254 from EM, Inc.; Flash chromatography (FC): silica gel 60 Å, 180-240 mesh from EM; NMR: ¹H (400.14 MHz), 13C (100.62 MHz) and 31P (161.99 MHz) spectra were recorded on Varian VNMR 400 spectrometer in CDCl3, DMSO-d6 or D2O as indicated below; ESI-MS spectra were run on Finningan LCQDUO; high resolution MS (FAB) spectra were carried out on JMS-700 MStation at the Mass Spectrometry Center of the University of Massachusetts (Amherst, Mass.).

Preparation of 3′-(N-tert-butyoxycarbonyl-L-phenylalanine)amino-3′-deoxyadenosine (Compound 2). To a solution of 3′-amino-3′-deoxyadenosine (1.07 g, 4.0 mmol) in 200 mL of anhydrous methyl sulfoxide, was added N-(tert-butyloxycarbonyl)-L-phenylalanine N-hydroxysuccinimide ester (1.81 g, 5.0 mmol) in 20 mL of anhydrous methyl sulfoxide. The solution was stirred at room temperature for 4 hours. A 10-mL volume of water was introduced and the solvent was evaporated under reduced pressure. The crude solid product was washed out by dichloromethane to give the desired product in the amount of 2.0 g (yield =97.3%).

Characterization: TLC (chloroform:methanol=85:15), R_{f}=0.52; ¹H NMR (DMSO-d₆): δ8.39 (s, 1H, C2-H), 8.14 (s, 1H, C8-H), 8.02 (d, J=7.7 Hz, 1H, C3′-NH), 7.32 (s, 2H, C6-NH₂), 7.28-m 7.15 (m, 5H, Ph), 6.93 (d, J=8.4 Hz), 1H, BocNH), 6.04 (d, J=3.3 Hz, 1H, C2′-OH), 5.94 (d, J=2.9 Hz, 1H, C1′-H), 5.17 (t, J=5.5 Hz, 1H, C5′-OH), 4.51 (br, 1H, C2′-H), 4.46 (m, 1H, C3′-H), 4.25 (m, 1H, CH), 3.90 (m, 1H, C4′-H), 3.66-3.43 (dm, 2H, C5′-H₂), 2.98-2.71 (dm, 2H, CH₂Ph), 1.28 (s, 9H, Bu^{t}). ¹³C NMR (DMSO-d₆) δ168.1, 152.2, 151.3, 148.7, 145.0, 135.3, 134.2, 125.4, 124.1, 122.3, 115.2, 85.4, 79.7, 74.2, 69.1, 57.2, 51.9, 46.5, 33.9, 24.2, 21.2. ESI-Mass (m/z): calculated. for C₂₄H₃₁N₇O₆: 513.2; found: 536.2 [M+Na]⁺.

Preparation of 5′-O-(tert-butyl-diphenylsilyl)-3′-(N-tert-butyloxycarbonyl-L-phenylalanine)amino-3′-deoxy-adenosine (Compound 3). To a solution of Compound 2 (1.2 g, 2.34 mmol) and 4-dimethylaminopyridine (286 mg, 2.34 mmol) in anhydrous pyridine (100 ml) was added tert-butyl-diphenylsilyl chloride (1.22 ml, 4.68 mmol). The mixture was stirred at room temperature for 2 days. Methanol (5 mL) was added and the mixture was stirred for 0.5 hour. After evaporation of solvent, the residue was dissolved in 300 mL of chloroform and washed with water (2×50 mL), saturated sodium bicarbonate (2×50 mL) and brine (2×50 mL). The combined aqueous layers were collected and extracted with chloroform (2×50 mL). The organic layers were dried over sodium sulfate and evaporated in vacuum. The crude product was purified by silica gel column eluted with dichloromethane/methanol (0-5%) to give a white solid (1.5 g; yield=85.4%).

Characterization: TLC (chloroform:methanol=95:5) R_{f}=0.22. ¹H NMR (CDCl₃): δ8.27 (s, 1H, C2-H), 8.03 (s, 1H, C8-H), 7.61=7.16 (m, 15H, 3 Ph), 6.50 (br, 1H, C3′-NH), 5.95 (br, 1H, C2′-OH), 5.74 (s, 2H, C6-NH₂), 5.63 (d, J=4.3 Hz, 1H, C1′-H), 5.23 (d, J=7.6 Hz, α-NH), 4.67 (br, 1H, C2′-H), 4.57 (m, 1H, C3′-H), 4.33 (m, 1H, CH), 4.14 (br, 1H, C4′-H), 3.88 (m, 2H, C5′-H₂), 3.03 (m, 2H, CH₂Ph), 1.40 (s, 9H, Bu^{t}), 0.95 (s, 9H, SiBu^{t}). ¹³C NMR (CDCl₃): δ172.1, 155.8, 152.8, 149.1, 138.9, 136.9, 136.0, 135.8, 133.1, 133.0, 130.2, 130.1, 129.5, 129.0, 128.1, 127.3, 120.2, 91.1, 85.0, 80.6, 74.8, 63.8, 56.4, 51.7, 39.2, 28.6, 27.1, 19.4. ESI-MS (m/z): calcld for C₄₀H₄₉N₇O₆Si 751.3, found 774.3 [M+Na]⁺.

Preparation of 5′-O-(tert-butyl-diphenylsilyl)-3′-(N-tert-butyloxycarbonyl-L-phenylalanine)amino-3′-deoxy-6-N,6-N,2′-O-tribenzoyl-adenosine (Compound 4). To a solution of Compound 3 (1.4 g, 1.86 mmol) in 80 mL of anhydrous pyridine at 0° C. was added dropwise benzoyl chloride (2.2 mL, 18.6 mmol). The mixture was stirred at 0° C. for 1 hour and 4 hours at room temperature. Ice-water (5 mL) was added and the mixture was allowed to stir for 0.5 hour at 0° C. and for another 3 hours at room temperature. The yellow solution was evaporated to dryness. The residue was dissolved in 400 mL of ethyl acetate and washed with water (2×50 mL), saturated sodium bicarbonate (2×50 mL) and brine (2×50 mL). The combined aqueous solution was extracted with chloroform (3×30 mL) and the organic layers were dried over sodium sulfate. Upon removal of solvents, the residue was chromatographed on flash column of silica gel, eluted with hexane/ethyl acetate (9:1 and 4:1) to give a white foam solid (1.96 g; yield=98.5%).

Characterization: TLC (hexane:ethyl acetate=1:1) R_{f}=0.71. ¹H NMR (CDCl₃): δ8.57 (s, 1H, C2-H), 8.27 (s, 1H, C8-H)), 8.07-7.07 (m, 30H, 6 Ph), 6.18 (d, J=2.0 Hz, 1H, C1′-H), 6.12 (d, J=8.2Hz, 1H, C3′-NH), 5.79 (dd, J=6.3,2.3 Hz, 1H, C2′-H), 5.32 (m, 1H, C3′-H), 5.00 (d, J=7.8 Hz, 1H, α-NH), 4.26 (m, 1H, CH), 4.00-3.85 (m, 3H, C4′-H, C5′-H₂), 3.40-2.86 (m, 2H, CH₂Ph), 1.31 (s, 9H, Bu^{t}), 1.05 (s, 9H, SiBu^{t}). ¹³C NMR (CDCl₃): δ172.5, 171.3, 165.2, 155.7, 152.7, 152.2, 143.8, 136.9, 136.0, 135.8, 134.4, 134.3, 133.7, 133.2, 132.8, 130.4-127.4 (m), 88.4, 84.3, 76.4, 63.5, 56.5, 49.5, 38.6, 28.4, 27.1, 19.4. ESI-MS (m/z):calcld for C₆₁H₆₁N₇O₉Si 1063.4, found 1086.3 [M+Na]⁺.

Deprotection of 5′-O-TBDPS: preparation of desired product, 3′-(N-tert-butyloxycarbonyl-L-phenylalanine)amino-3′-deoxy-6-N,6-N,2′-O-tribenzoyl-adenosine (Compound 5);3′-(N-tert-butyloxycarbonyl-L-phenylalanine)amino-3′-deoxy-6-N,6-N,2′-O,5′0-tetrabenzoyl-adenosine (Compound 6); and 3′-(N-tert-butyloxycarbonyl-L-phenylalanine)amino-3′-deoxy-6-N,2′-O-dibenzoyl-adenosine (Compound 7). To a solution of Compound 4 (730 mg, 0.69 mmol) in 70 ml of anhydrous tetrahydrofuran was added, at 0° C., 1M TBAF/THF solution (0.69 mL, 0.69 mmol). The mixture was stirred at 0° C. for 4 hours and at room temperature for 5 hours to yield a slightly yellow solution. Upon evaporation of solvent, the residue was dissolved in 200 mL of ethyl acetate and washed with saturated sodium bicarbonate (6×25 mL) and brine (50 mL). The combined aqueous solution was extracted with ethyl acetate (2×25 mL) and the collected organic layers were dried over sodium sulfate. Upon removal of solvent, the residue was loaded on a flash column of silica gel, and eluted with hexane/ethyl acetate (9:1 to 1:9) to give 20 mg of Compound 4 for a yield of 2.7%; 53 mg of Compound 7,3′-(N-tert-butyloxycarbonyl-L-phenylalanine)amino-3′-deoxy-6-N,6-N,2′-O,5′O-tetrabenzoyl-adenosine, for a yield of 8.3%; 322 mg of Compound 5,3′-(N-tert-butyloxycarbonyl-L-phenylalanine)amino-3′-deoxy-6-N,6-N,2′-O-tribenzoyl-adenosine for a yield of 56.8%; 67 mg of Compound 6,3′-(N-tert-butyloxycarbonyl-L-phenylalanine)amino-3′-deoxy-6-N,2′-O-dibenzoyl-adenosine for a yield of 13.5%; TLC [hexane:ethyl acetate=1:2] R_{f}=0.65; ESI-MS: calculated for C₅₂H₄₇N₇O₁₀-929.3; found-952.2 [M+Na]⁺; and other unidentified components.

Characterization of Compound 5: TLC (hexane:ethyl acetate=1:2), R_{f}=0.11. ¹H-NMR (CDCl₃): δ9.00 (s, 1H, C6-NHBz), 8.85 (s, 1H, C2-H), 8.30 (s, 1H, C8-H), 8.04-7.20 (m, 15H, 3 Ph), 6.49 (d, J=6.8 Hz, 1H, C3′-NH), 6.20 (d, J=3.2 Hz, 1H, C1′-H), 5.84 (dd, J=6.3, 3.5 Hz, 1H, C2′-H), 5.10 (dt, J=7.1, 6.8 Hz, 1H, C3′-H), 4.98 (br, 1H, α-NH), 4.58 (br, 1H, C5′-OH), 4.34 (dt, J=7.3, 7.1 Hz, 1H, CH), 4.14 (d, J=7.1 Hz, C4′-H), 4.04 (dbr, J=10.8 Hz, 1H, C5′-H), 3.79 (m, 1H, C5′-H′), 3.06 (m, 2H, CH₂Ph), 1.33 (s, 9H, Bu^{t}). ¹³C NMR (CDCl₃): δ172.6, 171.5, 165.3, 165.0, 155.9, 152.9, 151.2, 150.1, 142.4, 136.8, 134.3, 133.7, 133.1, 130.2, 129.4, 129.0, 128.9, 128.5, 128.3, 127.4, 123.9, 89.2, 84.8, 80.8, 76.5, 61.5, 56.4, 49.8, 38.4, 28.4; ESI-MS (m/z): calculated for C₃₈H₃₉N₇O₈ 721.3; found-744.3 [M+Na]⁺.

Preparation of 3′-(N-tert-butyloxycarbonyl-L-phenylalanine)amino-3′-deoxy-5′-O-(4,4′-dimethoxytrityl)-adenosine (Compound 10); 3′-(N-tert-butyloxycarbonyl-L-phenylalanine)amino-3′-deoxy-2′-O-(4,4′-dimethoxytrityl)-adenosine (Compound 8); 2′,5′-O-bis(4,4′-dimethoxytrityl)-3′-(N-tert-butyloxycarbonyl-L-phenylalanine)amino-3′-deoxy-adenosine (Compound 9). To a solution of Compound 2 (1.0 g, 1.95 mmol) in 20 mL of anhydrous pyridine, was added dimethoxytrityl chloride (0.7 g, 2.07 mmol). The mixture was stirred at room temperature for 2 days. A 1-mL volume of water was introduced and the mixture was stirred for 0.5 hour. The residue was flash chromatographed on silica gel, and eluted with gradient methanol (0-10%) in dichloromethane. Evaporation of the different portions gave 140 mg of starting material, Compound 2, at a yield of 14%; 750 mg of desired product, Compound 10, at a yield of 47.2%, and by-products, 3′-(N-tert-butyloxycarbonyl-L-phenylalanine)amino-3′-deoxy-2′-O-(4,4′-dimethoxytrityl)-adenosine (Compound 8); and 2′,5′-O-bis(4,4′-dimethoxytrityl)-3′-(N-tert-butyloxycarbonyl-L-phenylalanine)amino-3′-deoxy-adenosine (Compound 9). The mixture of Compound 8 and Compound 9 was stirred in 80% acetic acid solution (20 mL) at room temperature for 2 hours to regenerate the starting material, Compound 2. Upon evaporation of solvent in vacuo, the residue was purified on a silica gel column, eluted with gradient methanol (1-15%) in chloroform to re-obtain 300 mg of Compound 2.

Characterization of Compound 10: TLC (chloroform:methanol=9:1), R_{f}=0.26; ¹H NMR (CDCl₃): δ8.31 (s, 1H, C2-H), 8.05 (s, 1H, C8-H), 7.32-6.73 (m, 18H, Ph), 6.39 (br, 1H, C1′-H), 5.70 (sh, 1H, C2′-H), 5.65 (br, 2H, C6-NH₂), 5.15 (br, 1H,), 4.84 (br, 1H, ), 4.47 (br, 1H, ), 4.32-4.22 (m, 2H, ), 3.77 (s, 3H, OCH₃), 3.76 (s, 3H, OCH₃), 3.45-3.35 (m, 2H, C5′-H₂), 3.09-2.92 (m, 2H, CH₂Ph), 1.42 (s, 9H, Bu^{t}); ¹³C NMR (CDCl₃): δ172.5, 158.7, 156.0, 152.8, 148.8, 144.6, 138.8, 136.9, 135.9, 135.8, 130.4, 129.5, 128.9, 128.5, 128.1, 127.2, 127.1, 120.1, 113.4, 91.2, 86.7, 82.8, 80.4, 74.6, 63.4, 56.2, 55.4, 53.7, 51.5, 39.5, 28.5; ESI-Mass (m/z): calculated for C₄₅H₄₉N₇O₈-815.4; found 838.3 [M+Na]⁺.

Preparation of 3′-(N-tert-butyloxycarbonyl-L-phenylalanine)amino-3′-deoxy-5′-O-(4,4′-dimethoxytrityl)-6-N,6-N,2′-O-tribenzoyl-adenosine (Compound 11). A mixture of 740 mg (0.91 mmol) of Compound 10, 25 mL of anhydrous pyridine, and 1 mL (9.0 mmol) of benzoyl chloride was stirred at room temperature overnight and then stirred for 0.5 hour with 5 mL of water. After evaporation of solvent, the residue was dissolved in 150 mL of chloroform, washed with water (2×25 mL), sodium bicarbonate (2×25 mL) and brine (2×25 mL). The combined aqueous phases were extracted with chloroform (2×25 mL), and the combined organic layers were dried over sodium sulfate. After removal of solvent, the residue was purified on silica gel column, and eluted with gradient methanol (0-4%) in dichloromethane. Evaporation of solvent afforded 980 mg of Compound 11 as a white solid at a yield of 95.6%.

Characterization of Compound 11: TLC (chloroform:methanol=9:1), R_{f}=0.62. ¹H NMR (CDCl₃): δ8.61 (s, 1H, C2-H), 8.26 (s, 1H, C8-H), 7.92-6.78 (m, 28H, Ph), 6.20 (d, J=2.0 Hz, 1H, C1′-H), 5.92 (d, J=8.6 Hz, 1H, C3′-NH), 5.84 (dd, J=6.2, 2.0 Hz, 1H, C2′-H), 5.31 (dt, J=8.6, 6.0 Hz, 1H, C3′-H), 5.04 (d, J=7.4 Hz, 1H, NHCHCH₂), 4.21 (m, 1H, NHCHCH₂), 3.95 (br, 1H, C4′-H), 3.75 (s, 6H, OCH₃), 3.48 (m, 2H, NHCHCH₂), 3.04 (m, 1H, C5′H), 2.73 (m, 1H, C5′-H′), 1.32 (s, 9H, Bu^{t}); ESI-Mass (m/z): calculated for C₆₆H₆₁N₇O₁₁-1127.4; found-1150.4 [M+Na]⁺.

Preparation of 3′-(N-tert-butyloxycarbonyl-L-phenylalanine)amino-3′-deoxy-6-N,6-N,2′-O-tribenzoyl-adenosine (Compound 5). A mass of 1.36 g of Compound 11 (1.2 mmol) was stirred in an 80% acetic acid solution for 5 hours. After evaporation of solvent, the residue was dissolved in 200 mL of ethyl acetate, eluted with sodium bicarbonate (3×25 mL) and brine (2×25 mL). The combined aqueous layers were extracted with ethyl acetate (2×25 mL) and dried over sodium sulfate. After removal of solvent, the crude product was loaded on a flash column of silica gel and eluted with hexane/ethyl acetate (19:1 to 1:3). Evaporation in vacuo afforded 800 mg of a white solid at a yield of 80.4%.

Characterization of Compound 5: TLC (chloroform:methanol=9:1), R_{f}=0.52; ¹H NMR (CDCl₃): δ8.68 (s, 1H, C2-H), 8.43 (s, 1H, C8-H), 7.97-7.18 (m, 20H, Ph), 6.48 (d, J=3.3 Hz, 1H, C3′-NH), 6.22 (d, J=2.7 Hz, 1H, C1′-H), 5.75 (dd, J=6.1, 2.9 Hz, 1H, C2′-H), 5.10 (dt, J=7.0, 6.6 Hz, 1H, C3′-H), 4.95 (br, 1H, NH), 4.33 (dt, J=7.4, 7.0 Hz, 1H, CHCH₂Ph), 4.23 (br, 1H, C5′-OH), 4.11 (m, 1H, C4′-H), 4.02 (m, 1H, C5′H), 3.76 (m, 1H, C5′-H′), 3.05 (m, 2H, CH₂Ph), 1.32 (s, 9H, Bu^{t}); ¹³C NMR (CDCl₃): δ172.6, 172.5, 165.3, 152.6, 152.5, 152.3, 144.2, 136.7, 134.5, 134.2, 133.4, 130.2, 129.8, 129.4, 129.2, 129.1, 129.0, 128.5, 128.4, 127.5, 89.4, 84.8, 81.0, 76.7, 61.6, 56.3, 49.7, 38.2, 28.4; ESI-Mass (m/z): calculated for C₄₅H₄₃N₇O₉-825.3; found-848.3 [M+Na]⁺.

Preparation of 5′-DMTr-CpA-NHPheBoc. A mixture of 700 mg of Compound 5 (0.85 mmol) and 994 mg of a commercially available cytidine phosphoramidite, Compound 12 (1.03 mmol), was dried in vacuo overnight and dissolved in 8 mL of fresh distilled anhydrous acetonitrile. A mass of 361 mg of 1H-tetrazole (5.16 mmol) was dried overnight and dissolved in 12 mL of anhydrous acetonitrile, and then added to the above solution immediately. The mixture was stirred at room temperature for 1 hour. A 1.2-mL volume of tert-Butanol peroxide was added to the above reaction solution and stirring was continued for a half hour. After evaporation, the residue was dissolved in ethyl acetate (200 mL) and washed with water (2×20 mL), saturated sodium bicarbonate (2×20 mL) and brine (2×20 mL). The aqueous layers were collected together and extracted with ethyl acetate (3×20 mL). After removal of solvent, the residue was purified on a flash column of silica gel and eluted with gradient methanol in dichloromethane (0-2%). The excess C-block, Compound 12 was difficult to remove completely. The product obtained after chromatography was used for the next reaction without further purification. Characterization: ³¹P NMR (CDCl₃): −0.9, −0.7; ESI-Mass (m/z): calculated for C₉₁H₉₄N₁₁O₁₉PSi-1703.6; found-1726.2 [M+Na]⁺.

Preparation of 5′-HO-CpA-NHPheBoc (Compound 13). The product obtained as described above was stirred in 80% acetic acid (50 mL) for 3 hours and evaporated under vacuum. The residue was dissolved in ethyl acetate (200 mL) and washed with water (2×20 mL), saturated sodium bicarbonate (2×20 mL) and brine (2×20 mL). The aqueous layers were collected together and extracted with ethyl acetate (3×20 mL). After removal of solvent, the residue was purified on a flash column of silica gel and eluted with dichloromethane/ethyl acetate to give 850 mg of a white solid 850 mg at a yield of 75.2% (2 steps from Compound 5).

Characterization of Compound 13: ¹H NMR (CDCl₃): δ8.68 (s, 1H), 8.33 (s, 1H), 8.27-7.14 (m, 25H), 6.24 (br, 1H), 5.86 (br 1H), 5.60 (s, 1H), 5.15-3.70 (m, 13H), 3.02 (m, 2H), 2.70 (br, 2H), 1.20 (s, 9H), 0.88 (s, 9H), 0.12 (s, 3H), 0.10 (s, 3H); ³¹P NMR (CDCl₃): −0.7, −0.9; ESI-Mass (m/z): calculated. for C₇₀H₇₆N₁₁O₁₇PSi-1401.5; found-1424.1.

Preparation of 5′-DMTr-CpCpA-NHPheBoc (Compound 14). A mixture of 850 mg of Compound 13 (0.61 mmol) and 700 mg of C-block, Compound 12 (0.73 mmol) was dried in vacuo overnight and dissolved in 6 mL of fresh distilled anhydrous acetonitrile. A 256-mg mass of 1H-tetrazole (3.66 mmol) was dried overnight and dissolved in 8.5 mL of anhydrous acetonitrile, and then was added to the above solution immediately. After 1 hour of reaction at room temperature, more C-block, Compound 12 (174 mg, 0.19 mmol) was added and the reaction was stirred at room temperature for an additional 1 hour. A 1.2-mL volume of tert-Butanol peroxide was added to the above reaction solution and stirring continued for an additional half an hour. After evaporation, the residue was dissolved in ethyl acetate (200 mL) and washed with water (2×20 mL), saturated sodium bicarbonate (2×20 mL) and brine (2×20 mL). The aqueous layers were collected together and extracted with ethyl acetate (3×20 mL). After removal of solvent, the residue was passed through a silica gel column, and eluted with gradient dichloromethane/ethyl acetate (1:1). After removal of solvent, the solid containing a small amount of Compound 14 was used for the next step without further purification.

Characterization of Compound 14: ¹H NMR (CDCl₃): δ9.95, 9.94 (br, 1H), 8.96-6.88 (m, 47H), 6.39-3.54 (m, 24H), 3.81 (s, 6H), 3.76-3.54 (m, 4H), 3.05-2.84 (m, 2H), 2.74-2.53 (m, 4H), 1.11-0.83 (m, 27H), 0.20-0.02 (m, 12H); ¹³C NMR (CDCl₃): δ173.2, 172.9, 172.5, 172.4, 167.1, 165.7, 163.2, 159.0, 156.1, 155.7, 155.4, 152.5, 152.1, 144.5, 144.1, 137.3, 135.1, 135.0, 134.1, 133.2 (m), 130.4 (m), 129.6, 128.9, 128.4 (m), 127.6, 126.9, 116.8, 116.7, 113.6, 113.5, 97.7, 97.3, 90.1-86.8 (m), 82.2, 81.5, 80.4-79.8 (m), 75.9-72.9 (m), 68.3, 67.5, 66.4, 62.7 (m), 59.7, 55.4, 50.9, 40.4, 29.8, 28.0, 25.8, 19.7, 18.2, −4.6 (m); ³¹P NMR (CDCl₃): 0.7-−1.9 (m); ESI-Mass (m/z): calculated for C₁₁₆H₁₂₇N₁₅O₂₅P₂Si₂-2279.8; found-2303.4 [M+Na]⁺.

Preparation of 5′-HO-CpCpA-NHPheBoc (Compound 15). The product obtained according to the paragraph above was stirred in 80% acetic acid (50 mL) for 3 hours and evaporated under vacuum. The residue was dissolved in ethyl acetate (200 mL) and washed with water (2×20 ml), saturated sodium bicarbonate (2×20 ml) and brine (2×20 mL). The aqueous layers were collected together and extracted with ethyl acetate (3×20 mL). After removal of solvent, the residue was purified on a flash column of silica gel and eluted with dichloromethane/ethyl acetate to give 830 mg of a white solid at a yield of 68.6% (2 steps from Compound 13).

Characterization of Compound 15: ¹H NMR (CDCl₃): δ9.96, 9.94 (br, 1H), 8.68-7.12 (m, 40H), 6.39-3.85 (mbr, 23H), 3.04-2.88 (m, 2H), 2.74-2.48 (m, 4H), 1.10 (m, 9H), 0.90-0.80 (m, 18H), 0.10-0.01 (m, 12H); ¹³C NMR (CD₃OD): δ172.8, 172.5, 172.4, 167.2, 165.6, 163.5, 163.0, 156.3, 155.7, 152.6, 152.1, 146.1, 144.6, 137.1, 134.1, 133.9, 133.3, 130.2-127.8 (m), 127.0, 116.9, 116.8, 97.4, 91.8, 91.1, 89.1, 88.7, 83.1, 80.5, 78.9, 76.0, 75.1-73.2 (m), 68.4-65.5 (m), 65.9, 62.9, 60.6, 60.1, 59.4, 55.6, 50.4, 39.5, 28.1, 25.8, 19.7, 18.2, −4.6-−4.9 (m); ¹³P NMR (CDCl₃): 0.2-−1.3 (m); MS-FAB (m/z): calculated for C₉₅H₁₁₀N₁₅O₂₅P₂Si₂-1978.6839 [M+H]⁺; found-1978.6713.

Preparation of 3′-phenylalanineamide-3′-deoxy-pCpCpA (Compound I). A mixture of 100 mg of Compound 15 (0.05 mmol) in 16 mL of concentrated ammonia/ethanol (3:1) was sealed in a high pressure tube and stirred at 55° C. for 24 hours to give a colorless, clear solution. After evaporation under reduced pressure, the residue was loaded on a reversed-phase column, and eluted with water and water/methanol (3:1 and 1:1). After the removal of methanol in vacuo and freeze-drying, the reaction product resulted in 60 mg of a white solid, Compound 16 at a yield of 88.8%.

Characterization of Compound 16: ¹H NMR (CD₃OD): δ8.56 (s, 1H), 8.46 (br, 1H), 8.22 (s, 1H), 8.20 (sh, 1H), 7.31-7.20 (m, 5H), 6.06 (s, 1H), 5.95 (br, 1H), 5.86 (s, 1H), 5.85 (sh, d, 1H), 5.61 (s, 1H), 4.60-3.90 (m, 14H), 3.26 (m, 2H), 3.13 (m, 2H), 3.29 (m, 2H), 1.36 (s, 9H), 0.94 (s, 9H), 0.86 (s, 9H), 0.23 (s, 3H), 0.22 (s, 3H), 0.10 (s, 3H), 0.05 (s, 3H); ³¹P NMR (CD₃OD): 0.4, −0.2; ESI-Mass (m/z) calculated for C₅₄H₈₃N₁₃O₂₀P₂Si₂-1351.5; found-1350.4 [M-H]⁻, 1236.3.

The above product, Compound 16, was stirred in 3 mL of trifluoroacetic acid at 0° C. for 15 minutes. The orange reaction solution was evaporated to dryness and co-evaporated twice with acetonitrile (10 mL) at low temperature until complete removal of trifluoroacetic acid. The crude product was used for the next reaction without further purification.

Characterization: ¹H NMR (CD₃OD): δ6.85 (br, 1H), 8.56 (br, 1H), 8.39 (br, 1H), 8.24 (br, 1H), 7.32 (m, 7H), 6.17 (br, 1H), 6.08 (br, 2H), 5.82 (br, 1H), 5.73 (m, 1H), 4.74-3.60 (mbr, 25H), 3.20 (br, 4H), 0.90 (s, 9H), 0.87 (s, 9H), 0.18 (s, 3H), 0.16 (s, 3H), 0.15 (sh, 3H), 0.10 (s, 3H); ³¹P NMR (CD₃OD): −0.3 (br); ESI-Mass (m/z) calculated for C₄₉H₇₅N₁₃O₁₈P₂Si₂-1251.4; found-1250.3 [M-H]⁻.

The above solid was treated with 1 mL of a mixed solution of triethylamine/triethylamine trifluoric acid/1-methyl-pyrollidinone (1.5 mL: 2 mL: 3 mL) at 65° C. for 1.5 hours. After evaporation in vacuo, the residue was dissolved in water (10 mL) and washed with chloroform (3×5 mL). The aqueous layer was concentrated to 2 mL and loaded on a reversed-phase column, and eluted with water and water/methanol (4:1 and 3:2). After removing methanol under reduced pressure, the aqueous solution was lyophilized to give 30 mg of a white solid, Compound I (61.1% from 15).

Characterization of Compound I: ¹H NMR (D₂O): δ8.43 (s, 1H), 8.23 (s, 1H), 7.93 (d, J=8.0 Hz, 1H), 7.86 (d, J=8.0 Hz, 1H), 7.46-7.28 (m, 5H), 5.98 (d, J=3.2 Hz, 1H), 5.94 (d, J=8.0 Hz, 1H), 5.85 (d, J=8.0 Hz, 1H), 5.74 (m, 2H), 4.61-3.80 (m, 16H), 3.31-3.15 (m, 2H); ³¹P NMR (D₂O): 0.2, 0.0 MS-FAB (m/z) calculated for C₃₇H₄₈N₁₃O₁₈P₂-1024.2716 [M+H]⁺; found 1024.2969.

### EXAMPLE 2

Assays of Peptidyl Transferase Activity

Preparation of Labled 5′-OH-CCA-NH-Phe. The 5′-OH-CCA-NH-Phe (Compound I), prepared according to the procedure of Example 1, was labeled with ³²P. The kinase reaction was carried out with 1 nmol 5′-OH-CCA-NH-Phe, 50 μCu γ-³²P-ATP, and 20 units of T4-polynucleokinase in the presence of 1×PNK buffer at 37° C. for one hour. Excess ATP (100 nmol) was added into the radioactive mix and the labeling reaction was continued for another one hour to convert all 5′-OH-CCA-NH-Phe into 5′-³²P-CCA-NH-Phe. The reaction mixture was then loaded onto 24% non-denaturing polyacrylamide gel for purification. The labeled Compound I (³²P-CCA-NH-Phe) was detected by film and the gel containing ³²P-CCA-NH-Phe was cut out, squeezed by syringe and soaked in pure water at 4° C. overnight. The soaking solution was separated from the gel by centrifugation and loaded on reverse phase C₁₈ cartridge. The C₁₈ cartridge was pre-conditioned by 5 mL CH₃CN, 5 ml CH₃CN:H₂O and 5 mL H₂O. Soaking solution was allowed to flow through the column by gravity. Then the bound ³²P-CCA-NH-Phe was eluted by 1 mL CH₃CN:CH₃OH:H₂O (35:35:30). After being dried by lyophilization, ³²P-CCA-NH-Phe was dissolved in 100 μL distilled water and stored at −20° C.

Ribosome Preparation. Ribosomes and ribosomal subunits were prepared as described in the literature (Noll, M.; et al., J. Mol. Biol. 75: 281-294 (1975)). Bacterial strain E. Coli MRE 600 was harvested at a density of A₆₅₀=0.5. Cell pellets were resuspended in buffer B (20 mM HEPES, pH 7.6, 5 mM MgCI₂, 30 mM NH₄Cl, 2 mM spermidine, and 5 mM 2-mercaptoethanol). Cells were broken by grinding with autoclaved alumina (1 g cell paste/2 gram alumina) in a pre-cooled mortar until a smooth, thick and sticky paste was obtained and “popping” sounds were heard. The cell paste was extracted by slowly adding buffer B (2 mL per gram of cell paste) and continued grinding. After a few minutes, RNase-free DNase (4 g/mL) was added and grinding was continued for a few more minutes until a reduction in viscosity is observed. Cell debris and alumina were cleared by centrifugation twice at 30,000 g for 30 minutes. The S30 supernatant was layered on a 1.1 M sucrose cushion made in buffer B and centrifuged at 50,000 g in a Beckman 50.2 Ti rotor at 4° C. After 19 hours centrifugation, crude ribosomes were obtained as a transparent pellet. To get 70S ribosomes, the ribosome pellet was washed and resuspended in buffer D (same as buffer B except that the MgCl₂ concentration is 10 mM) and stored at −80° C. For continuous preparation of the ribosomal subunits, the pellet was dissolved in buffer C (20 mM HEPES, pH 7.6, 1 mM MgCl₂, 300 mM NH₄Cl, 2 mM spermidine, and 5 mM 2-mercaptoethanol) with gentle stirring at 4° C. for 2 hours. The solution was clarified by low-speed centrifugation. Sixty A₂₆₀ units of the ribosomes were loaded on 34 mL 10-30% sucrose gradient made in buffer C. Centrifugation was performed at 43,000 g in a SW28 rotor at 4° C. for 17 hours. Fractions of the sucrose gradient were monitored by UV absorbance at A₂₆₀ and the corresponding 50S and 30S peaks were collected. Ribosome subunits were precipitated by ethanol and stored in buffer B at −80° C.

Peptidyltransferase Activity Assay. The standard assay of peptidyl transferase activity was performed with 22 μM 50S subunit, 50 μM CCA-Met-Biotin, trace amount of ³²P-CCA-NH-Phe (˜5×10⁻⁴ μM) in the presence of 50 mM Tris•HCl (pH 7.5), 35 mM MgCl₂, 100 mM NH₄Cl and 1000 mM KCl at 37° C. A 1-μL aliquot of the reaction was taken out at specific time points, quenched with 2 μL of a quench buffer (formamide +0.05% xylene cyanol) and loaded on 24%/7.5 M urea polyacrylamide denaturing gel. For reaction validation, samples were incubated with 10 μg streptavidin at room temperature for 15 minutes prior to being loaded on the gel. The reaction product was quantitated by a Molecular Dynamics PhosphorImager and the fraction of the product relative to the total reactant was plotted against different time points. The observed rate constants were obtained by curve fit using KaleidaGraph.

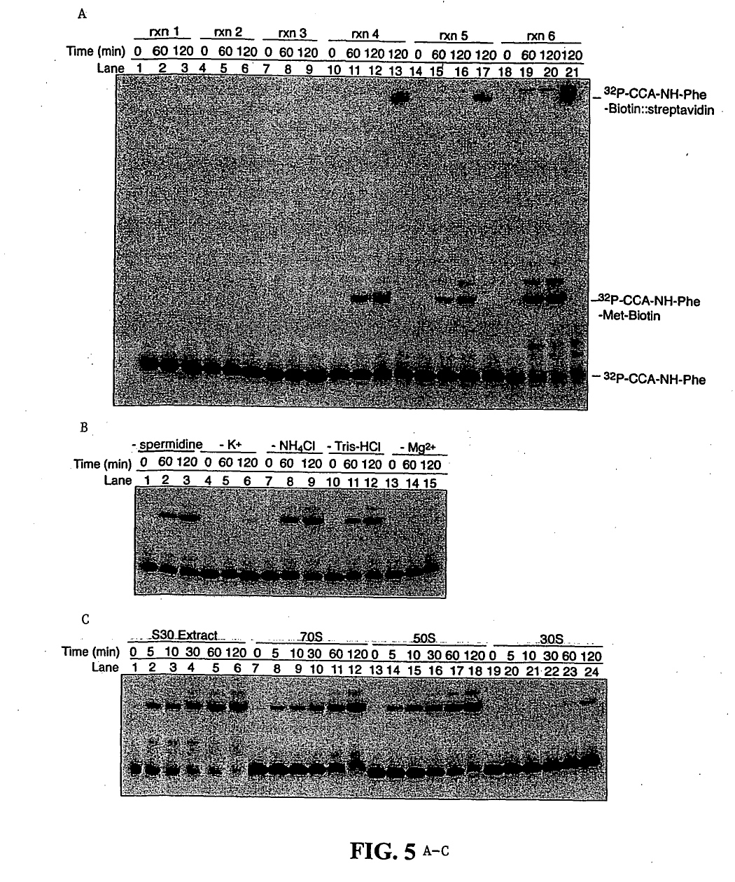

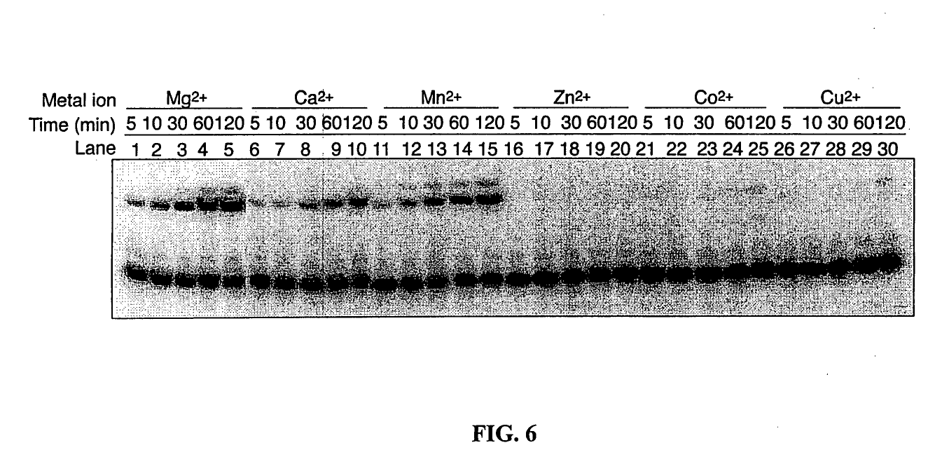

Peptidyltransferase activity with CCA-NH-Phe and CCA-Met-Biotin. Peptidyltransferase activity with CCA-NH-Phe and CCA-Met-Biotin was initially tested in S30 extract, which is the crude extract prior to the preparation of the ribosomes. If these two analogs are active substrates for the ribosome, the free amine on phenylalanine will attack the carbonyl carbon on methionine and a peptide bond will be formed between the two amino acids (FIG. 5). The formed dipeptide can be separated from the reactant and observed on a 24%/7.5 M urea polyacrylamide gel. FIG. 6 is the typical gel showing the peptidyltransferase activity with the two CCA substrates.

Reactions were performed in S30 extract with 50 μM CCA-Met-Biotin, trace amount of ³²P-CCA-NH-Phe (˜5×10−4 μM) in the presence of 50 mM Tris•HCl (pH 7.5), 5 mM MgCl₂, 10 mM NH₄Cl, 100 mM KCl and 1.5 mM spermidine at 37° C. The lower band corresponds to labeled ³²P-CCA-NH-Phe; the middle band is the formed dipeptide (³²P-CCA-NH-Phe-Met-Biotin) labeled by biotin; the highest band represents ³²P-CCA-NH-Phe-Met-Biotin::streptavidin complex. When only ³²P-CCA-NH-Phe was included in the reaction (FIG. 5A, lanes 1-3), there is no product formed. When 32P-CCA-NH-Phe was incubated with either S30 extract (FIG. 5A, lanes 4-6) or CCA-Met-Biotin (FIG. 5A, lanes 7-9), there is still no product observed. However, peptide bond formation was observed when both ³²P-CCA-NH-Phe and CCA-Met-Biotin were incubated with S30 extract (FIG. 5A, lanes 10-12). Product was also observed when the two substrates were incubated with 70S (FIG. 5A, lanes 14-16) and CCA-Met-Biotin was replaced by tRNA-Met-Biotin (FIG. 5A, lanes 18-20). Upon streptavidin incubation, the product was shifted to the top of the gel (FIG. 5A, lanes 13, 17, and 21). Together, these results demonstrate that ³²P-CCA-NH-Phe and CCA-Met-Biotin are active substrates for the ribosome and a peptide bond is formed between the two substrates.

Buffer and salt requirements for the peptidyltransferase activity were examined. Assays were performed with ³²P-CCA-NH-Phe, CCA-Met-Biotin and 70S in the absence of different components. Without spermidine, NH₄Cl or Tris•HCl, the ribosome was still active (FIG. 5B, lanes 1-3, 7-9, 10-12). However, both Mg²⁺ and K⁺ are required for 70S in catalyzing the peptidyltransfer reaction between the two substrates (FIG. 5B, lanes 4-6, 13-15). Peptidyltransferase activity was also tested with different concentrations of Mg²⁺, K^{+ and NH}⁴⁺ (data not shown). The optimized condition for the peptidyltransferase activity is reached with 35 mM MgCl₂, 100 mM NH₄Cl, 1000 mM KCl in the presence of 50 mM Tris•HCl.

Under the optimized reaction conditions, peptidyltransferase activity with different ribosomal components were compared (FIG. 5C). Activity was found in the S30 extract, 70S, and 50S, but no activity was observed in the 30S.

Metal Ion Studies. Metal ions are required for the peptidyltransferase reaction as woulkd be recognized by one of skill in the appropriate art. With two novel substrates described in the present invention, the type of metal ion required was examined. Six kinds of divalent metal ions (Mg²⁺, Ca²⁺, Mn²⁺, Zn²⁺, Co²⁺, Cu²⁺) were tested for ribosomal peptidyl transferase activity. Generally, 5 mM Mg²⁺, Ca²⁺, Mn²⁺, Zn²⁺, Co²⁺, or Cu²⁺ was included in the standard reaction condition to examine peptidyl transferase activity. For metal ion concentration dependence, standard reactions were carried out with 10 to 100 mM Mg²⁺, Ca²⁺, or Mn²⁺.

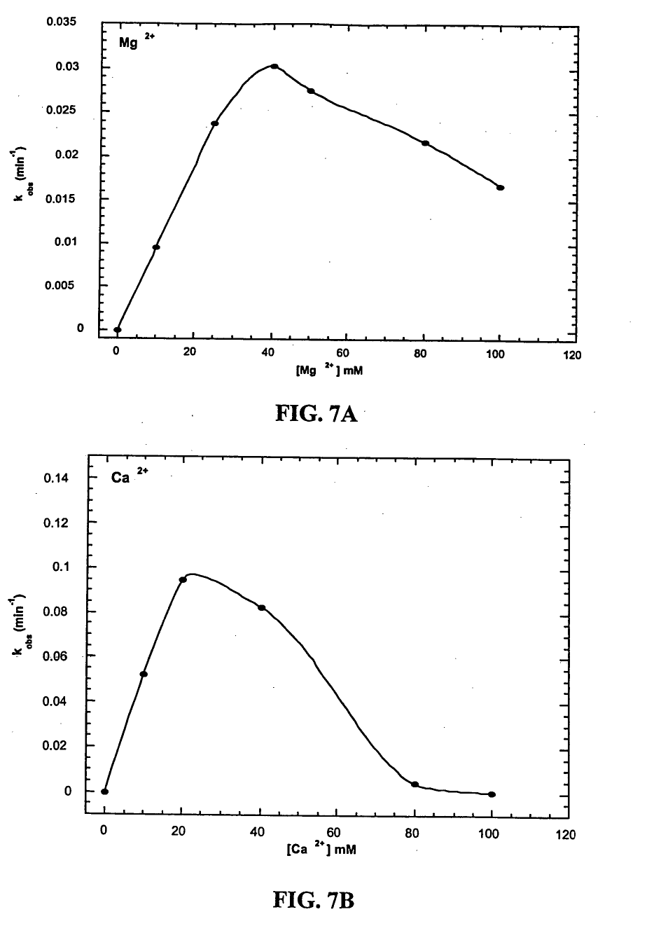

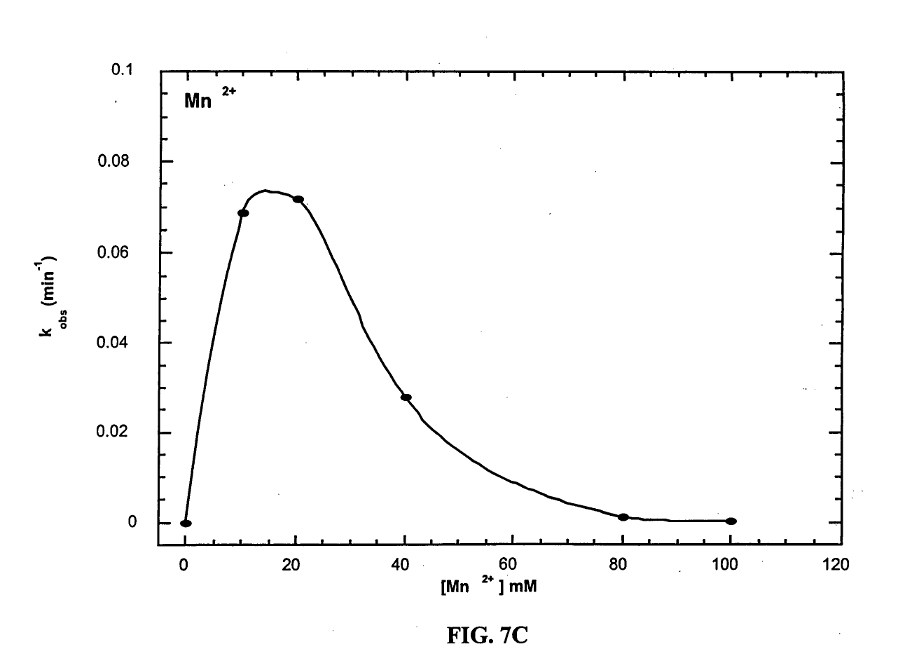

Reactions were performed with 22 μM 50S subunit (1 A₂₆₀ unit), 50 μM CCA-Met-Biotin, trace amount of ³²P-CCA-NH-Phe in the presence of 50 mM Tris•HCl (pH 7.5), 100 mM NH₄Cl, 1000 mM KCl and 5 mM divalent metal ion at 37 ° C. 50S is active with Mg²⁺, Ca²⁺, or Mn²⁺ alone (FIG. 6, lanes 1-15); however, it is not active with Zn²⁺, Co²⁺, or Cu²⁺ alone (FIG. 6, lanes 16-30). 50S activity was also tested at different concentrations of divalent metal ions (FIGS. 7A, 7B, and 7C). In the presence of Mg²⁺, Ca²⁺, or Mn²⁺, the activity increased with concentrations at low concentration; however, the reaction was inhibited at high concentrations of divalent metal ions.

Kinetic studies. Generally, kinetic studies were performed with the 50S subunit or S30 extract, trace amount of ³²P-CCA-NH-Phe in the presence of 50 mM Tris•HCl (pH 7.5), 35 mM MgCl₂, 100 mM NH₄Cl and 1000 mM KCl with various concentrations of CCA-Met-Biotin. Determination of k_{cat} and Kₘ was achieved by fitting data into the Michaelis-Menten equation.

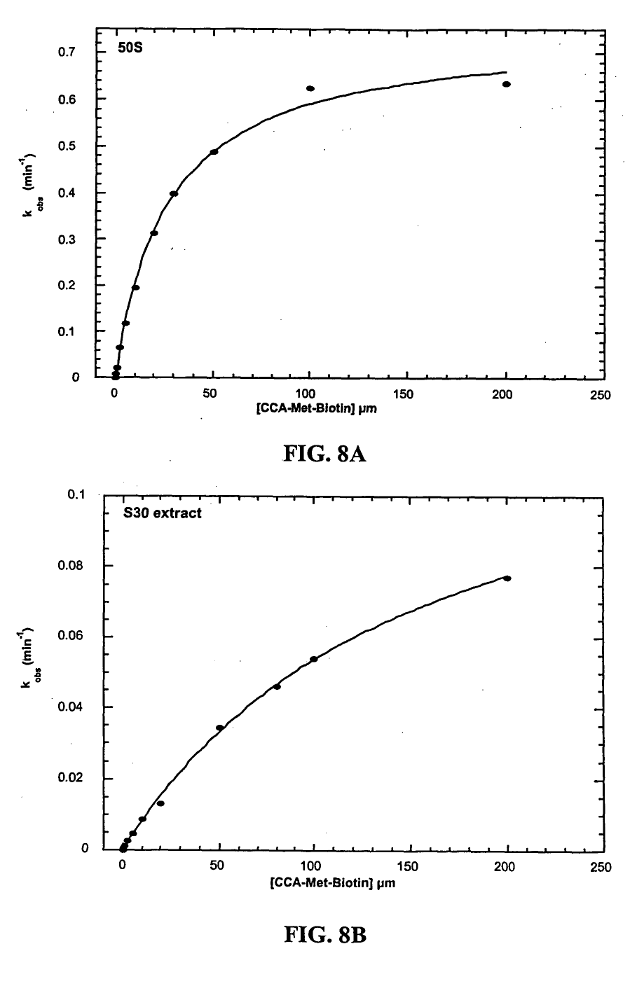

For example, a kinetics study of the peptidyl transferase reaction was performed by varying the concentrations of CCA-Met-Biotin, fixing the concentration of 50S (22 μM), and including a trace amount of ³²P-CCA-NH-Phe (˜5×10⁻⁴ μM). The best-fit data derived from Michaelis-Menten equations were k_{cat}=0.75 min⁻¹ and Kₘ=26.3 μM (FIG. 8A). Kinetic studies was also performed in S30 extract and the obtained values were k_{cat}=0.14 min⁻¹ and Kₘ=158.4 μM (FIG. 8B). Kₘ in S30 extract is 6-fold higher than that in 50S, indicating that CCA-Met-Biotin has higher affinity in binding with 50S than with S30 extract.

pH Dependence and Solvent Isotope Effects. Generally, the pH dependence was investigated under standard reaction conditions at different pH's (Bis•Tris propane, pH 6.4-9.5; MES, pH 5.5-6.3). To examine pH dependence with different divalent metal ions, standard reactions were performed with 20 mM Mg²⁺, Ca²⁺, or Mn²⁺ at different pHs. The observed rate constants were plotted against pH and data were fit by KaleidaGraph.

For solvent isotope experiments, all buffers were made in D₂O, lyophilized and re-dissolved in D₂O (repeated twice) to remove any hydrogen atoms. Calculation of pD was determined by adding 0.4 to a pH meter reading. Salts, ribosomes and two substrates were dried by lyophilization and dissolved in 99.9% D₂O and this process was repeated twice. The pD dependence experiments were performed in the same manner as the pH dependence experiments.

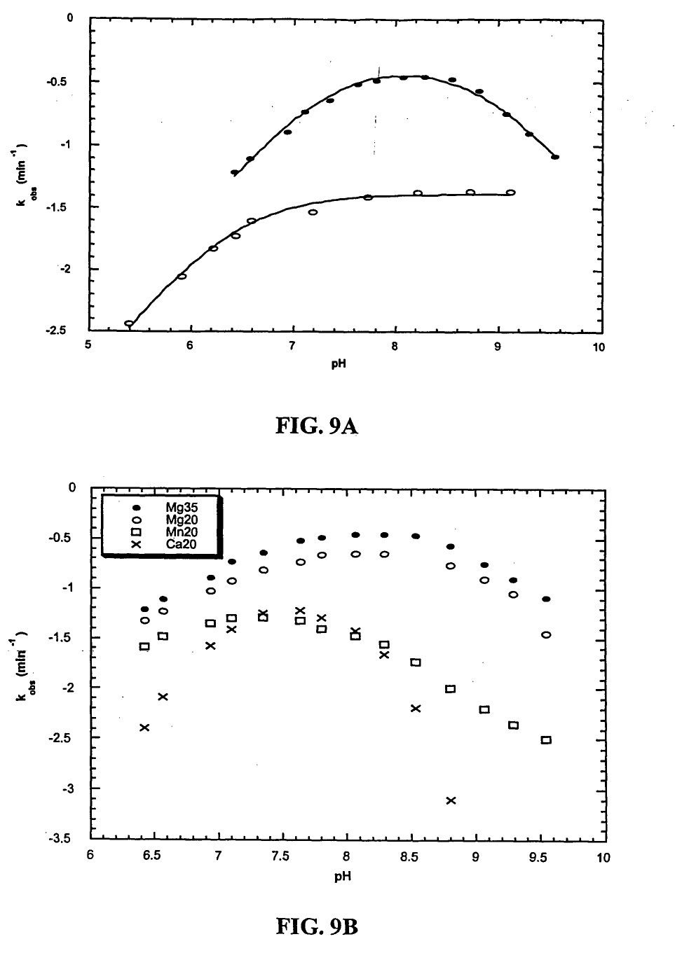

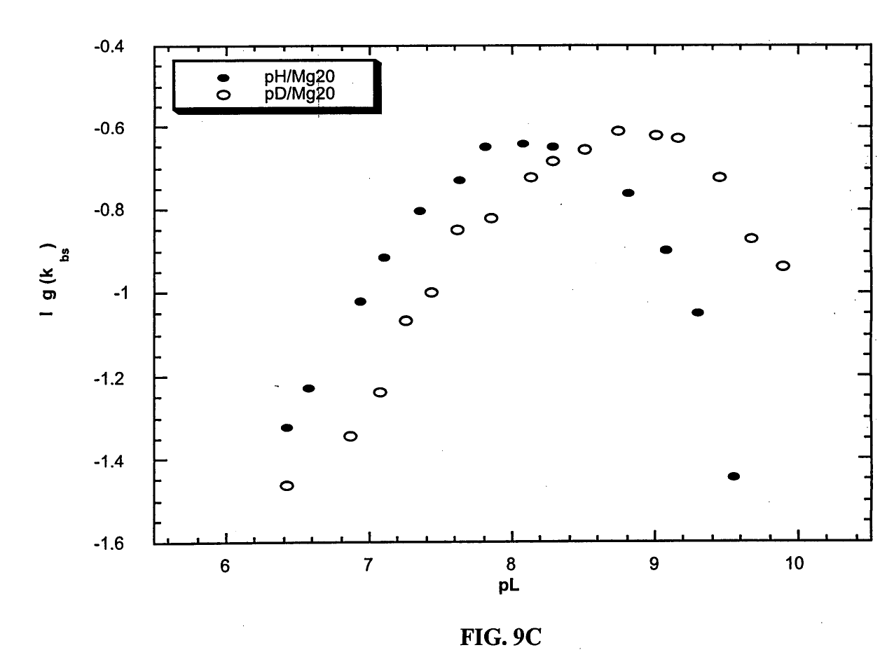

By way of example, the pH dependence of the peptidyltransferase reaction was examined with 50S and S30 extract (FIG. 9A). When the peptidyltransferase activity was measured with the 50S subunit, the pH profile gave a bell-shape curve from pH 6.42 to 9.54 with the highest activity reached at pH 8.07. Two pKₐ values were obtained from the pH profile, with pKₐ₁=7.29 and pKₐ₂=8.88. However, a different pH profile was observed when the reaction was performed with S30 extract. From pH 5.39 to pH 6.58, the profile showed a linear relationship between log (k_{obs}) and pH; and the observed rate constant was insensitive to high pHs (pH 7.72-9.12). The slopes of the linear part of the pH profiles at low pHs are 0.70 for S30 and 0.64 for 50S.

In addition, the pH dependence was observed in the presence of various divalent metal ions. Reactions were carried out with 22 μM 50S subunit, 50 μM CCA-Met-Biotin, Biotin, trace amount of ³²P-CCA-NH-Phe (˜5×10⁻⁴ μM) 100 mM NH₄Cl, 1000 mM KCl and 20 mM of a divalent metal ion (Mg²⁺, Ca²⁺, or Mn²⁺) at different pHs (6.42-9.54). FIG. 9B shows the pH dependence of 50S-catalyzed peptidyltransferase reaction with Mg²⁺, Ca²⁺, or Mn²⁺. Different pH profiles were observed with Mg²⁺, Ca²⁺, or Mn²⁺.

Solvent isotope effects were also examined for the 50S-catalyzed peptidyltransferase reaction. pD-dependence was observed in the presence of 20 mM Mg²⁺and compared with the pH-dependence in 20 mM Mg²⁺. As shown in FIG. 9C, the pD profile shifted with respect to the pH profile. Similar activity was achieved at pH 8.07 and pD 8.74. The peptidyltransferase activity was lower in D₂O than in H₂O at low pHs (6.42-8.28); however, at high pHs (8.50-9.89), the activity was higher in D₂O than in H₂O.

### OTHER EMBODIMENTS

A number of embodiments of the invention have been described. Nevertheless, it will be understood that various modifications may be made without departing from the spirit and scope of the invention. Accordingly, other embodiments are within the scope of the following claims.

What is claimed is:

1. A compound of formula (I),
X-CpCpA-NH-Phe (I)
wherein,
X is absent or a label.

2. The compound of claim 1, wherein the label is a radioactive label.

3. The compound of claim 1, wherein the label is a fluorescence label.

4. The compound of claim 1, wherein the label is attached to the 5′-hydroxy group.

5. The compound of claim 1, wherein the label is ³²P.

6. A method of making a compound of formula (I) in claim 1, the method comprising providing a compound of formula (II): 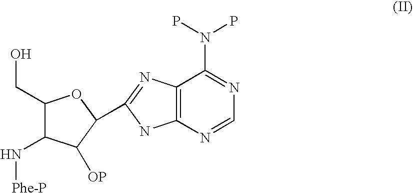
wherein
each P is independently an oxygen- or nitrogen-protecting group;
and converting it to a hydroxyl-group protected derivative of formula (III): 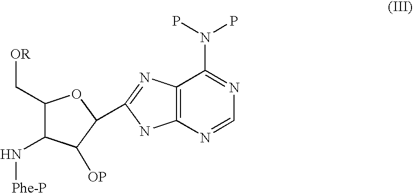
wherein
each R is an oxygen-protecting group; and
each P is independently an oxygen- or nitrogen-protecting group.

7. The method of claim 6, wherein R is a silyl group.

8. The method of claim 6, wherein R is tert-butyldiphenylsilyl or dimethoxytrityl.

9. The method of claim 6, wherein P is benzyl.

10. The method of claim 6, further comprising converting the hydroxyl-group protected derivative to a compound of formula (I).

11. The method of claim 6, further comprising removing the protecting group from the hydroxyl-group protected derivative and attaching a label to the resulting hydroxyl-group.

12. The method of claim 11, further wherein the label is attached by reaction of γ-³²P-ATP with the resulting hydroxyl-group.

13. The method of claim 11, wherein the label is a radioactive label.

14. The method of claim 11, wherein the label is ³²P.

15. A method for monitoring peptide bond formation, the method comprising:
providing a mixture comprising a peptidyl transferase, a peptidyl-tRNA analog, and an aminoacyl-tRNA analog of formula (I) in claim 1;
incubating the mixture under conditions sufficient to enable peptide bond formation; and
monitoring the mixture for peptide bond formation.

16. The method of claim 15, wherein the aminoacyl-tRNA analog is ³²p*CpCpA-NH-Phe.

17. The method of claim 15, wherein the mixture further comprises a test compound, and the method is used to monitor the effect of the test compound on peptide bond formation.

18. The method of claim 15, wherein the peptidyl-tRNA analog is an amino acid conjugated to an oligonucleotide.

19. The method of claim 15, wherein the peptidyl-tRNA analog is 5′-CCA-phenylalanine, 5′-CACCA-phenylalanine, 5′-CACCA-methionine, 5′-CAACCA-formylmethionine, or tRNA-phenylalanine.

20. The method of claim 15, wherein the aminoacyl-tRNA analog is capable of being detected by polyacrylamide gel electrophoresis (PAGE).

21. The method of claim 15, wherein the peptidyl transferase is a ribosomal subunit.

22. A method for screening test compounds, comprising:
(a) incubating a mixture comprising a peptidyl transferase, a peptidyl-tRNA analog, an aminoacyl-tRNA analog of formula (I) in claim 1, and one or more test compounds; and
(b) determining the rate of transfer of the peptidyl moiety of the peptidyl-tRNA analog to the free amino group of the aminoacyl-tRNA analog for each test compound.

23. The method of claim 22, wherein the screening is performed in a high-throughput format.

24. The method of claim 22, wherein the test compounds are members of a combinatorial library.

25. The method of claim 22, wherein the peptidyl transferase is a ribosomal subunit.

26. A method for determining whether a test compound is a candidate antibacterial agent, the method comprising:
(a) incubating a mixture comprising a peptidyl transferase, a peptidyl-tRNA analog, an aminoacyl-tRNA analog of formula (I) in claim 1, and one or more test compounds;
(b) determining the rate of transfer of the peptidyl moiety of the peptidyl-tRNA analog to the free amino group of the aminoacyl-tRNA analog for each test compound; and
(c) identifying one or more test compounds that inhibit peptidyl transferase activity, wherein a compound that inhibits peptidyl transferase activity is a candidate antibacterial agent.

27. A kit comprising a compound of formula (I) in claim 1 and instructions for using the compound in an assay to determine peptidyl transferase activity.

28. An inhibitor of peptidyl transferase activity identified by a method comprising:
(a) incubating a mixture comprising a peptidyl transferase, a peptidyl-tRNA analog, an aminoacyl-tRNA analog of formula (I) of claim 1, and one or more test compounds, under conditions allowing transfer of the peptidyl moiety of the peptidyl-tRNA analog to the aminoacyl-tRNA analog;
(b) determining the rate of transfer of the peptidyl moiety of the peptidyl-tRNA analog to the free amino group of the aminoacyl-tRNA analog for each test compound; and
(c) identifying one or more test compounds that inhibit peptidyl transferase activity, wherein a test compound so identified is an inhibitor of peptidyl transferase activity.
